(19) United States
(12) Patent Application Publication
(10) Pub. No.: US 2017/0298297 A1
(43) Pub. Date: Oct. 19, 2017

(54) LIPOSOMES-CONTAINING ANTIFOULING COMPOSITIONS AND USES THEREOF

(71) Applicant: Yeda Research and Development Co. Ltd., Rehovot (IL)

(72) Inventors: Jacob KLEIN, Rehovot (IL); Ronit GOLDBERG, Rehovot (IL); Weifeng LIN, Rehovot (IL); Sabrina JAHN, Rehovot (IL)

(21) Appl. No.: 15/516,407

(22) PCT Filed: Oct. 1, 2015
(86) PCT No.: PCT/IL2015/050987

§ 371 Date: Apr. 2, 2017

(30) Foreign Application Priority Data
Oct. 1, 2014 (IL) 234929

(51) Int. Cl. C11D 3/00; C11D 3/36; C11D 3/22; G02C 7/04

(52) U.S. Cl. CPC C11D 3/0078; G02C 7/04; C11D 3/222; C11D 3/225; C11D 3/365

(57) ABSTRACT

A method of inhibiting adsorption of a biofouling-promoting agent on a surface of a substrate and a method of inhibiting biofilm formation on a surface of a substrate are disclosed herein. The methods comprise comprising contacting the substrate with a composition which comprises liposomes. Further disclosed herein is a composition comprising liposomes, identified for use in inhibiting adsorption of a biofouling-promoting agent on a surface of a substrate and/or inhibiting biofilm formation on a surface of a substrate. Further disclosed are articles comprising a substrate and a composition described herein being applied on a surface of the substrate, and a method of treating keratitis in a contact lens-wearing subject, by rinsing and/or immersing a contact lens in a solution comprising liposomes.

### FIELD AND BACKGROUND OF THE INVENTION

The present invention, in some embodiments thereof, relates to material science and, more particularly, but not exclusively, to compositions and methods for reducing biofilm formation on a surface of various substrates.

Fouling of surfaces by adsorption of unwanted material from fluids with which they are in contact can lead to highly undesirable effects. The fouling material may consist of a non-living substance or living organisms. Fouling by microorganisms, such as bacteria, may pose a health threat.

A biofilm is a group of microorganisms, such as bacteria or fungi, in which cells stick to each other on a surface. The adherent cells are frequently embedded within a self-produced matrix composed of extracellular DNA, proteins and polysaccharides. Microbial cells in a biofilm are physiologically distinct from free, individual cells in a liquid medium, which are referred to as “planktonic” cells.

Biofilm formation on a substrate may be facilitated by a “conditioning film”, formed by adsorption of various substances, such as proteins and polysaccharides, onto the substrate surface. Microorganisms may then be capable of adhering to the substrate to a far greater extent than in the absence of the conditioning film.

In addition to posing potential health threats, biofilms may also promote corrosion. By acting as a diffusion barrier which reduces local concentrations of oxygen, biofilms can accelerate the corrosion of a metallic substrate by creating a differential aeration concentration cell. The corrosion and weathering caused by biofilm can lead to considerable damage to heat exchangers, unexpected corrosion of stainless steel, and premature destruction of membranes, and many other technological, industrial and homestead aliments.

Infections associated with medical devices in contact with human tissue (especially implanted devices such as stents or catheters) typically begin with the adhesion of bacteria to a surface, and their subsequent proliferation, resulting in a biofilm, in which the bacteria become highly antibiotic-resistant. For example, neutralizing bacteria in a biofilm may require a 1000-fold dose of antibiotic compared to planktonic bacteria [Kostina et al., Biomacromolecules 2012, 13:4164-4170]. Such infections are frequent—approximately 4% of all implanted vascular grafts and medical heart valves, 2% of implanted joint prostheses and 5% of fracture fixation devices become infected [Kostina et al., Biomacromolecules 2012, 13:4164-4170]. The cost of curing such infections may exceed 50,000$ per case, apart from suffering or morbidity. Catheters in particular are often colonized by bacteria, with resultant infection, suffering and costs. Such infections represent about half of all hospital associated infections (HAI), and are especially costly and difficult to treat.

Efforts have been made to develop materials which exhibit reduced protein adhesion, with the goal of reducing adhesion of microbes that form biofilms, as microbial adhesion is mediated by surface proteins.

Hydrogels formed by copolymerization of carboxybetaine monomers with 2-hydroxyethyl methacrylate (HEMA) have been reported to exhibit low levels of fouling from blood plasma [Kostina et al., Biomacromolecules 2012, 13:4164-4170]. Modification of polysulfone (PSI) membranes with 2-methacryloxyethyl phosphorylcholine (MPC) polymer was reported to decrease protein adsorption [Ishihara et al., Biomaterials 1999, 20:1553-1559]. Other studies have associated zwitterionic hydrogel surfaces with enhanced antifouling properties [Schlenoff, Langmuir 2014, 30:9625-9636; Yang et al., J Mat Chem B 2014, 2:577-584].

Coatings have been described for reducing biofilm formation. In one example, a polymeric antifouling coating was based on aggregation of a short amphiphilic four armed PEG-dopamine polymer into particles and on surface binding by catechol chemistry [Mizrahi et al., Langmuir 2013, 29:10087-10094]. Another example is surface grafting of a brush of poly(oligoethylene glycol methyl ether acrylate), which was reported to improve the antifouling properties of poly(2-hydroxyethyl methacrylate-co-methyl methacrylate) [Bozukova et al., Langmuir 2008, 24:6649-6658].

Contact lenses frequently adsorb proteins from the eye fluid, which limits their long-term use. The accumulated protein can lead to discomfort and eye infection, as bacterial adhesion to soft contact lenses and biofilm formation have been reported to be associated with keratitis [Bourcier et al., Br J Ophthalmol 2003, 87:834-838; Tam et al., Invest Ophthalmol Vis Sci 2010, 51:3100-3106].

Tran et al. [Cont Lens Anterior Eye 2012, 35:155-162] describes an attempt o modulate adhesion of P. aeruginosa to silicone-hydrogel contact lenses by enhancing wettability of the lens surface using poloxamer or ethylene oxide-butylene oxide surfactants. The surfactants were reported to enhance wettability, but no correlation was found between P. aeruginosa adhesion and contact lens wettability.

International Patent Application PCT/IL2014/050604 describes hydrogels with liposomes dispersed therein, which exhibit a reduced friction coefficient compared to neat hydrogels.

U.S. Provisional Patent Application 62/012,379 describes aqueous solutions comprising an ionic polymer and liposomes, for rinsing and/or immersing therein a contact lens, and/or for reducing a friction coefficient of a surface. U.S. Provisional Patent Application 62/012,379 further describes methods of reducing a friction coefficient of a surface by attaching an ionic polymer to the surface and contacting the ionic polymer with liposomes, thereby coating the surface with an amphiphilic lipid.

International Patent Application Publication WO 2008/038292 and U.S. Patent Application Publication No. 20100098749 describe a use of liposomes of several phospholipids above their liquid-crystalline-phase to gel-phase transition temperature (Tm) for lubrication of joints.

Additional background art includes Chen et al. [Polymer 2010, 51:5283-5293].

### SUMMARY OF THE INVENTION

The present inventors have now surprisingly uncovered that liposome-containing compositions, when combined with a hydrogel, may be efficiently utilized for reducing the absorption of biofouling-promoting agents to surfaces and hence can be used for inhibiting biofilm formation on the surface of hydrogel-containing substrates or can be combined with a hydrogel for inhibiting biofilm formation on surfaces of substrates which are not hydrogels.

According to an aspect of some embodiments of the invention there is provided a method of inhibiting adsorption of a biofouling-promoting agent on a surface of a substrate, the method comprising contacting the substrate with a composition which comprises liposomes.

According to an aspect of some embodiments of the invention there is provided a method of inhibiting biofilm formation on a surface of a substrate, the method comprising contacting the substrate with a composition which comprises liposomes.

According to some of any of the embodiments of the invention relating to a method of inhibiting adsorption and/or biofilm formation, the method is for reducing adhesion of pathogenic microorganisms to a medical device.

According to an aspect of some embodiments of the invention there is provided a composition comprising liposomes, identified for use in inhibiting adsorption of a biofouling-promoting agent on a surface of a substrate.

According to an aspect of some embodiments of the invention there is provided a composition comprising liposomes, identified for use in inhibiting biofilm formation on a surface of a substrate.

According to an aspect of some embodiments of the invention there is provided an article comprising a substrate and the composition comprising liposomes, as described herein, being applied on a surface of the substrate.

According to some of any of the embodiments of the invention, the article is characterized in that upon contact with the agent under biofouling-promoting conditions, a biofilm load on the substrate is lowered by at least 10% compared to a biofilm load on the substrate when being devoid of the composition, upon contact with the agent under the same conditions.

According to some of any of the embodiments of the invention, the article is characterized in that under biofouling-promoting conditions, a biofilm load on the substrate is lowered by at least 10% compared to a biofilm load on the substrate when being devoid of the composition, under the same conditions.

According to some of any of the embodiments of the invention, the article is, or forms at least a portion of, a medical device.

According to some of any of the embodiments of the invention relating to inhibition of adsorption of a biofouling-promoting agent, the adsorption of the biofouling-promoting agent is reduced by at least 30% relative to adsorption on the surface of the substrate in the absence of the composition which comprises liposomes.

According to some of any of the embodiments of the invention relating to inhibition of adsorption of a biofouling-promoting agent, the biofouling-promoting agent is selected from the group consisting of a biofouling-promoting protein and a biofouling-promoting polysaccharide.

According to some of any of the embodiments of the invention, the substrate is composed of a hydrogel.

According to an aspect of some embodiments of the invention there is provided a method of inhibiting adsorption of a biofouling-promoting agent on a surface of a substrate which is composed of a hydrogel, the method comprising contacting the substrate with a composition which comprises liposomes.

According to some of any of the embodiments of the invention, the contacting comprises incorporating the composition which comprises liposomes within the hydrogel.

According to some of any of the embodiments of the invention, the substrate is prepared by forming the hydrogel in the presence of the liposomes. According to some of any of the embodiments of the invention, the substrate is prepared by forming the hydrogel in the presence of a solution of a polymer.

According to some of any of the embodiments of the invention, the composition further comprises a hydrogel.

According to some of any of the embodiments of the invention, the substrate does not comprise a hydrogel.

According to an aspect of some embodiments of the invention there is provided a method of inhibiting adsorption of a biofouling-promoting agent on a surface of a substrate which does not comprise a hydrogel, the method comprising contacting the substrate with a composition which comprises liposomes.

According to some of any of the embodiments of the invention, the composition is prepared by forming the hydrogel in the presence of the liposomes.

According to some of any of the embodiments of the invention, forming the hydrogel is such that the liposomes are dispersed throughout the bulk of the hydrogel.

According to some of any of the embodiments of the invention, the composition is prepared by forming the hydrogel in the presence of a solution of a polymer.

According to some of any of the embodiments of the invention, forming the hydrogel is such that the polymer is dispersed throughout the bulk of the hydrogel.

According to some of any of the embodiments of the invention, the composition is in a form of a hydrogel comprising the liposomes.

According to some of any of the embodiments of the invention, the liposomes are dispersed throughout the bulk of the hydrogel.

According to some of any of the embodiments of the invention, the composition further comprises a polymer dispersed throughout the bulk of said hydrogel.

According to some of any of the embodiments of the invention, inhibition of the adsorption of the biofouling-promoting agent is substantially unchanged after at least one dehydration-rehydration cycle of the hydrogel.

According to some of any of the embodiments of the invention, inhibition of the biofilm formation is substantially unchanged after at least one dehydration-rehydration cycle of said hydrogel.

According to some of any of the embodiments of the invention, the composition which comprises liposomes is a liquid comprising the liposomes, and the contacting comprises rinsing and/or immersing at least a portion of the substrate in the liquid.

According to some of any of the embodiments of the invention, the biofilm formation is reduced by at least 10% relative to biofilm formation on the surface of the substrate in the absence of the composition which comprises liposomes.

According to some of any of the embodiments of the invention, the composition which comprises liposomes further comprises a polymer.

According to some of any of the embodiments of the invention, the polymer is a non-cross-linked polymer.

According to some of any of the embodiments of the invention, the polymer is a hydrophilic polymer.

According to some of any of the embodiments of the invention, the hydrophilic polymer is selected from the group consisting of poly(2-hydroxyethyl methacrylate), hydroxypropyl methyl cellulose and an ionic polymer.

According to some of any of the embodiments of the invention, the hydrophilic polymer is hydroxypropyl methyl cellulose.

According to some of any of the embodiments of the invention, the liposomes are selected from the group consisting of small unilamellar vesicles (SUV), large unilamellar vesicles (LUV) and multilamellar vesicles (MLV).

According to some of any of the embodiments of the invention, the liposomes comprise at least one phosphatidylcholine.

According to some of any of the embodiments of the invention, the liposomes are characterized by a melting point below a temperature of the surface during the contacting of the liposomes with the surface.

According to some of any of the embodiments of the invention, the liposomes are characterized by a melting point below 37° C.

According to some of any of the embodiments of the invention, the composition is in a form of a liquid comprising the liposomes.

According to some of any of the embodiments of the invention, the contacting comprises coating the surface with an amphiphilic lipid of the liposomes.

According to some of any of the embodiments of the invention, the biofilm is a biofilm of a microorganism selected from the group consisting of a bacterium, an archaeal microorganism, a fungus, and a protist.

According to an aspect of some embodiments of the invention there is provided a method of inhibiting biofilm formation on a contact lens, the method comprising rinsing and/or immersing the contact lens in a solution comprising liposomes and an aqueous carrier.

According to some of any of the embodiments of the invention relating to a contact lens, the liposomes are characterized by a melting point below 36° C.

According to some of any of the embodiments of the invention, the biofilm is a biofilm of a pathogenic microorganism associated with keratitis.

According to an aspect of some embodiments of the invention there is provided a method of treating keratitis in a contact lens-wearing subject in need thereof, the method comprising rinsing and/or immersing a contact lens in a solution comprising liposomes and an aqueous carrier, prior to insertion of said contact lens in an eye of the subject. According to some of any of the embodiments of the invention, the abovementioned solution in which a contact lens is rinsed and/or immersed further comprises at least one polymer.

According to some of any of the embodiments of the invention, the at least one polymer is a hydrophilic polymer.

According to some of any of the embodiments of the invention, the carrier of the solution in which a contact lens is rinsed and/or immersed is an ophthalmically acceptable carrier.

According to some of any of the embodiments of the invention, the abovementioned contact lens comprises a hydrogel surface. According to some of any of the embodiments of the invention, the hydrogel comprised by the abovementioned contact lens comprises a polymer selected from the group consisting of a poly(2-hydroxyethyl methacrylate), a polyvinyl alcohol and a silicone.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a bar graph showing the relative adsorption of a protein (anti-IgG antibody) to a cross-linked poly(2-hydroxyethyl methacrylate) hydrogel (Neat HEMA) and to cross-linked poly(2-hydroxyethyl methacrylate) hydrogels loaded with DMPC (dimyristoyl phosphatidylcholine) liposomes (+DMPC) or HSPC (hydrogenated soy phosphatidylcholine) liposomes (+HSPC), as well as with alginate, hyaluronic acid (HA), hydroxypropyl methyl cellulose (HPMC), polyvinylpyrrolidone (PVP) or polyethylene oxide (PEO), or without a polymer loaded into the hydrogel (WO polymer) (normalized to adsorption to a tissue culture polystyrene surface);

FIG. 2 is a bar graph showing the relative adsorption of a protein (anti-IgG antibody) to a poly(2-hydroxyethyl methacrylate) hydrogel (Neat HEMA) cross-linked with 1%, 2% or 4% EDGMA (ethylene glycol dimethacrylate), and to poly(2-hydroxyethyl methacrylate) hydrogels cross-linked with 1%, 2% or 4% EDGMA and loaded with non-cross-linked poly(2-hydroxyethyl methacrylate) (+polyHEMA polymer), with DMPC (dimyristoyl phosphatidylcholine) multilamellar vesicles (+MLV DMPC), and with DMPC multilamellar vesicles mixed with non-cross-linked poly(2-hydroxyethyl methacrylate) (+polyHEMA/DMPC) (normalized to adsorption to a tissue culture polystyrene (TCPS) surface);

FIG. 3 is a bar graph showing the relative adsorption of a protein (anti-IgG antibody) to a cross-linked poly(2-hydroxyethyl methacrylate) hydrogel (Neat HEMA) and to cross-linked poly(2-hydroxyethyl methacrylate) hydrogels loaded with DMPC (dimyristoyl phosphatidylcholine) (+DMPC) in the form of multilamellar vesicles (+MLV) or small unilamellar vesicles (+SUV) (normalized to adsorption to a tissue culture polystyrene (TCPS) surface);

FIG. 4 is a bar graph showing the relative adsorption of a protein (anti-IgG antibody) to a methacrylic acid-polyethylene oxide hydrogel (MAA-PEO neat) and to a methacrylic acid-polyethylene oxide hydrogel loaded with dimyristoyl phosphatidylcholine liposomes (MAA-PEO DMPC) (normalized to adsorption to a tissue culture polystyrene (TCPS) surface);

FIG. 5 is a bar graph showing the relative adsorption of a protein (anti-IgG antibody) to a gelatin methacrylate hydrogel (Gelatin-MA neat) and to a gelatin methacrylate hydrogel loaded with dimyristoyl phosphatidylcholine liposomes (Gelatin-MA+DMPC) (normalized to adsorption to a tissue culture polystyrene (TCPS) surface);

FIG. 6 is a bar graph showing the relative adsorption of a protein (anti-IgG antibody) to a cross-linked poly(2-hydroxyethyl methacrylate) hydrogel (Neat HEMA) and to cross-linked poly(2-hydroxyethyl methacrylate) hydrogels loaded with DMPC (dimyristoyl phosphatidylcholine) liposomes (+DMPC) or HSPC (hydrogenated soy phosphatidylcholine) liposomes (+HSPC), as well as with alginate, hyaluronic acid (HA), hydroxypropyl methyl cellulose (HPMC), polyvinylpyrrolidone (PVP), polyethylene oxide (PEO) or gelatin, or without a polymer loaded into the hydrogel (WO polymer) (normalized to adsorption to a tissue culture polystyrene surface; hydrogels were completely dried at 37° C. and then rehydrated in PBS prior to protein adsorption);

FIG. 7 is a bar graph showing the relative adsorption of a protein (anti-IgG antibody) to a poly(2-hydroxyethyl methacrylate) hydrogel (Neat HEMA) cross-linked with 1%, 2% or 4% EDGMA (ethylene glycol dimethacrylate), and to poly(2-hydroxyethyl methacrylate) hydrogels cross-linked with 1%, 2% or 4% EDGMA and loaded with non-cross-linked poly(2-hydroxyethyl methacrylate) (+polyHEMA polymer), with DMPC (dimyristoyl phosphatidylcholine) multilamellar vesicles (+MLV DMPC), and with DMPC multilamellar vesicles mixed with non-cross-linked poly(2-hydroxyethyl methacrylate) (+polyHEMA/DMPC Mixture) (normalized to adsorption to a tissue culture polystyrene (TCPS) surface; hydrogels were completely dried at 37° C. and then rehydrated in PBS prior to protein adsorption);

FIG. 8 is a bar graph showing the relative adsorption of a protein (anti-IgG antibody) to a cross-linked poly(2-hydroxyethyl methacrylate) hydrogel (Neat HEMA) and to cross-linked poly(2-hydroxyethyl methacrylate) hydrogels loaded with DMPC (dimyristoyl phosphatidylcholine) (+DMPC) in the form of multilamellar vesicles (+MLV) or small unilamellar vesicles (+SUV) (normalized to adsorption to a tissue culture polystyrene (TCPS) surface; hydrogels were completely dried at 37° C. and then rehydrated in PBS prior to protein adsorption);

FIG. 9 is a bar graph showing the relative adsorption of a protein (anti-IgG antibody) to narafilcon A contact lenses following treatment by being immersed in PBS or in a solution of hyaluronic acid (HA), dimyristoyl phosphatidylcholine liposomes (MLV DMPC), or both hyaluronic acid and dimyristoyl phosphatidylcholine liposomes (HA+DMPC); and

FIG. 10 is a bar graph showing the relative adsorption of a protein (anti-IgG antibody) to nelfilcon, filcon II and methafilcon A contact lenses following treatment by being immersed in PBS or in a solution of hydroxypropyl methyl cellulose (HPMC), dimyristoyl phosphatidylcholine liposomes (DMPC), or both hydroxypropyl methyl cellulose and dimyristoyl phosphatidylcholine liposomes (HPMC+DMPC).

### DESCRIPTION OF SPECIFIC EMBODIMENTS OF THE INVENTION

The present invention, in some embodiments thereof, relates to material science and, more particularly, but not exclusively, to compositions and methods for reducing biofilm formation on a surface of various substrates.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

As discussed hereinabove, the formation of biofilms presents a formidable challenge in medicine and other practices.

The present inventors have now surprisingly uncovered that combining liposomes and hydrogel-containing matrix reduced absorption of biofouling-promoting agents and thus imparts anti-biofouling (ABF) properties to the matrix. The present inventors have therefore envisioned that including liposomes in a hydrogel-containing substrate and/or using a composition composed of liposomes and a hydrogel on non-hydrogel substrates can be used for reducing the absorption of biofouling-promoting agents to the surface of such substrates and to inhibit biofilm formation on such surfaces. These findings represent a novel solution of the challenge of inhibiting biofilm formation on a variety of articles, such as medical, agricultural and veterinary articles.

As demonstrated in the Examples section that follows, treatment of substrates with liposomes was surprisingly found to considerably reduce adsorption of biofouling-promoting agents such as proteins, in a simple and efficient manner. Protein adsorption is associated with biofilm formation because protein adsorption can create a conditioning film on a substrate which facilitates cell attachment, and/or because proteins are an important component of cells.

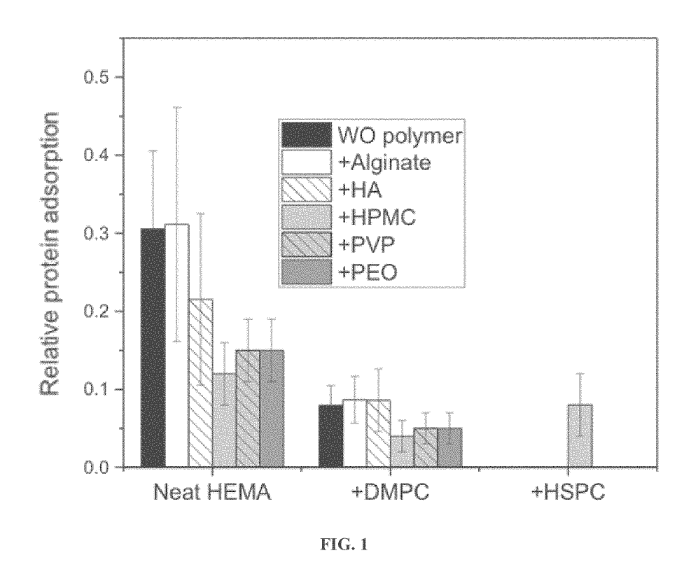

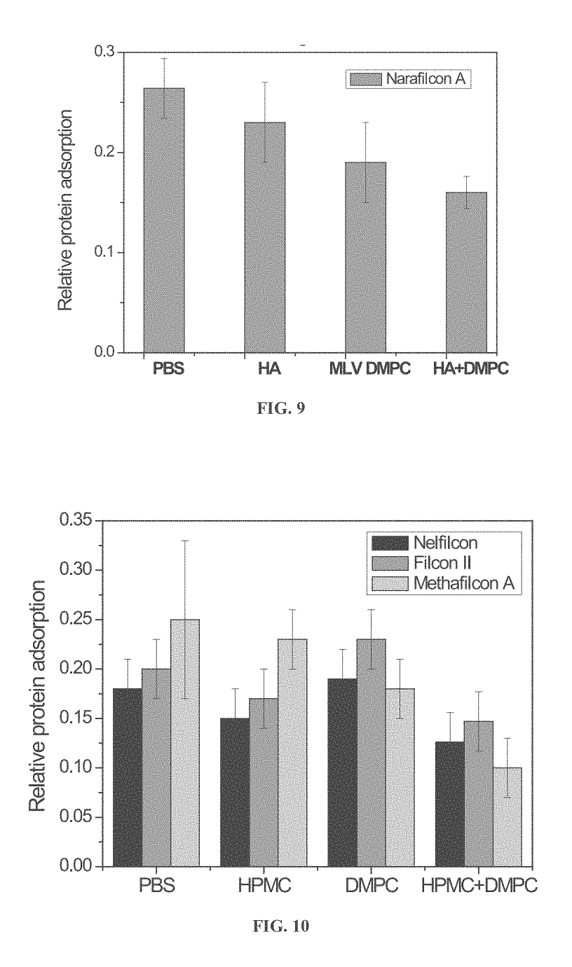

Referring now to the drawings, FIGS. 1-8 show that loading various hydrogels with liposomes surprisingly inhibits protein adsorption to the hydrogel surface. FIGS. 9 and 10 show that simple immersion of contact lenses in a solution comprising liposomes inhibits protein adsorption to the contact lens surface.

In addition, FIGS. 1, 9 and 10 further show that addition of a polymer to liposomes in the hydrogel or solution surprisingly enhances the inhibition of protein adsorption. Such an effect is particularly surprising, as it would have been expected that a surface comprising liposome lipids and a polymer would have properties intermediate between those of a surface comprising lipids alone and a surface comprising the polymer alone, rather than anti-adsorption properties superior to those of either the lipid-comprising surface and the polymer-comprising surface.

These results indicate that contact of a substrate with liposomes can efficiently reduce adsorption to a surface, as well as biofouling. The results further indicate that the effect of the liposomes can optionally be enhanced by use of the liposomes in combination with a polymer. It is to be appreciated that such methodology is simple to perform and efficacy was demonstrated for liposomes and polymers which are naturally occurring and/or recognized as non-toxic. Thus, the methodology can advantageously be utilized in a wide variety of applications, including applications wherein the substrate is implantable or otherwise contacts a body.

Without being bound by any particular theory it is believed that lipids from the liposomes promote the formation of hydration layers at the surface, which serves as a barrier to adsorption on the surface. It is further believed that inclusion of liposomes in a hydrogel results in lipids reaching the surface by diffusion, and that immersion of a substrate (e.g., contact lens) in a solution comprising liposomes results in lipids from the solution becoming adhered to the surface.

Based on the results presented herein, inhibition of adsorption and biofilm formation on of a wide variety of substrates may be effected, in accordance with various embodiments of the invention described herein.

Inhibition of Adsorption:

According to an aspect of some embodiments of the invention, there is provided a method of inhibiting adsorption of a biofouling-promoting agent on a surface of a substrate. The method, according to some embodiments of the present invention, is effected by contacting the substrate with a composition which comprises liposomes.

Herein, the term “contacting” encompasses effecting any form of physical contact between two or more substances (e.g., a substrate and composition described herein), including mixing, incorporating and/or embedding one substance within another, and contact only at an interface between the substances. The term “contacting” further encompasses forming one or more of the substances in a manner such that it is in contact with the other substance(s) as soon as it is formed, as well as contacting pre-formed substances.

The term “biofouling-promoting agent”, as used herein throughout, refers to an agent whose presence facilitates formation and/or participates in formation of a biofilm (as defined herein) on a substrate surface. An agent is considered to facilitate formation of a biofilm on a substrate surface when a presence of the agent enhances formation of a biofilm on a substrate surface as compared to formation of a biofilm on the same substrate surface in an absence of the agent. An agent is considered to participate in formation of a biofilm on a substrate surface when the biofilm formed on the surface comprises the agent as a portion of the biofilm.

In some embodiments of any of the embodiments described herein, an agent is identified as a biofouling-promoting agent by comparing growth (e.g., over the course of 1, 2, 3, 4, 5, 6 or 7 days) of a biofilm (e.g., P. aeruginosa) on the surface in the presence of an aqueous liquid (e.g., water or broth, optionally at 37° C.) and the agent, to growth of a biofilm (under the same conditions) on the surface in the presence of the same aqueous liquid (e.g., water or broth) without the agent. The agent is optionally mixed within the aqueous liquid, or alternatively, adsorbed onto the surface prior to exposure of the surface to the aqueous liquid. The growth of the biofilm is considered as the biofilm load at the end of the growth period (e.g., 1, 2, 3, 4, 5, 6 or 7 days) minus the initial biofilm load. Optionally, the measurement is performed such that the initial biofilm is substantially zero (e.g., absent or at least undetectable), for example, the microorganism is in a planktonic form, such that growth of the biofilm is considered as the biofilm load at the end of the growth period. In some embodiments of any of the embodiments described herein, biofilm load is defined as an area of the biofilm.

In some embodiments of any of the embodiments described herein, biofilm load is defined as a mass and/or volume of the biofilm.

In some embodiments of any of the embodiments described herein, biofilm load is defined as a number of cells in the biofilm.

The biofilm load may optionally be determined using any technique known in the art for detecting and quantifying an amount of cells and/or microorganisms in a biofilm.

In some of these embodiments, an agent is considered a biofouling-promoting agent if the growth of a biofilm in its presence is at least 10% higher than growth of a biofilm in the absence of the agent.

In some of these embodiments, an agent is considered a biofouling-promoting agent if the growth of a biofilm in its presence is at least 20% higher than growth of a biofilm in the absence of the agent.

In some of these embodiments, an agent is considered a biofouling-promoting agent if the growth of a biofilm in its presence is at least 50% higher than growth of a biofilm in the absence of the agent.

In some of these embodiments, an agent is considered a biofouling-promoting agent if the growth of a biofilm in its presence is at least 100% higher than (i.e., two-fold) the growth of a biofilm in the absence of the agent.

Examples of biofouling-promoting agents include, without limitation, a biofouling-promoting protein and a biofouling-promoting polysaccharide, that is, any protein or polysaccharide which is a biofouling-promoting agent as defined herein.

In some embodiments of any of the embodiments described herein, the biofouling-promoting agent is a protein.

In some embodiments of any of the embodiments described herein, the method is considered as being capable of inhibiting adsorption of a biofouling-promoting agent when the method is capable of inhibiting adsorption of a selected biofouling-promoting agent (e.g., the selected agent is considered representative of biofouling-promoting agents in general). In some embodiments, the selected biofouling-promoting agent is a protein. In some embodiments, the selected protein is an antibody which does not exhibit any specific affinity to the substrate (e.g., an anti-IgG antibody, as exemplified herein).

The term “biofilm”, as used herein throughout, refers to an aggregate of living cells which are stuck to each other and/or immobilized onto a surface as colonies. The cells are frequently embedded within a self-secreted matrix of extracellular polymeric substance (EPS), also referred to as “slime”, which is a polymeric sticky mixture of nucleic acids, proteins and polysaccharides.

In the context of the present embodiments, the living cells forming a biofilm can be cells of a unicellular microorganism, including prokaryotes (e.g., bacteria, archaeal microorganisms) and eukaryotes such as fungi and protists (e.g., algae, euglena, protozoa, dinoflagellates, apicomplexa, trypanosomes, amoebae) and the like; or cells of multicellular organisms, in which case the biofilm can be regarded as a colony of cells (as in the case of the unicellular organisms) or as a lower form of a tissue.

According to some embodiments of any of the embodiments of the present invention, the cells are of microorganism origins, and the biofilm is a biofilm of microorganisms, such as bacteria, archaeal microorganisms, protists and fungi. The cells of a microorganism growing in a biofilm are typically physiologically distinct from cells in the “planktonic” form of the same organism, which by contrast, are single cells that may float or swim in a liquid medium.

The term “substrate” as used herein, is as described herein, and further refers to any surface, structure, product or material which can support, harbor or promote the growth of a microorganism. The substrate is optionally a portion of an object (e.g., an article of manufacture) which can support, harbor or promote the growth of a microorganism. Such a portion of an object may span only a portion of an area of the object, such that a surface of the substrate represents only a portion of a surface of the object (e.g., a portion most likely to support, harbor or promote the growth of a microorganism); and/or span only a portion of the thickness of the object (e.g., along an axis perpendicular to a surface of the substrate and object), such that the substrate does not include the entire volume of the object lying underneath a surface of the substrate (which may represent the entire surface of the object or only a portion of the surface of the object). Non-limiting examples include the inner walls of a storage container (e.g., a box, a can) and/or conduit (e.g., a tubes, a pipe) for an organic product susceptible to spoilage associated with biofouling, for example, food and/or drink (e.g., a food container, a water pipe), surfaces intended to come into contact with such an organic product (e.g., agricultural and/or food processing machinery, a kitchen surface, water-purification equipment), and surfaces exposed to moisture (e.g., bathroom walls, water system components, outer surfaces of housing exposed to rain, surfaces in the vicinity of water leakage).

In some embodiments, the substrate is a medical device or any other device which is intended for contacting a living tissue, as defined herein.

As used herein, the phrase “medical device” includes any material or device that is used on, in, or through a subject's body, for example, in the course of medical treatment (e.g., for a disease or injury). The subject may be human or a non-human animal, such that the phrase “medical device” encompasses veterinary devices. Medical devices include, but are not limited to, such items as medical implants (including permanent implants and transient implants), wound care devices, drug delivery devices, contact lenses and body cavity and personal protection devices. The medical implants include, but are not limited to, catheters (e.g., urinary catheters, intravascular catheters), injection ports, intubation equipment, dialysis shunts, wound drain tubes, skin sutures, vascular grafts, implantable meshes, intraocular devices, heart valves, and the like. Wound care devices include, but are not limited to, general wound dressings, biologic graft materials, tape closures and dressings, and surgical incise drapes. Drug delivery devices include, but are not limited to, needles, drug delivery skin patches, drug delivery mucosal patches and medical sponges. Body cavity and personal protection devices, include, but are not limited to, tampons, sponges, surgical and examination gloves, and toothbrushes. Birth control devices include, but are not limited to, intrauterine devices (IUDs), diaphragms and condoms.

In some embodiments of any of the embodiments described herein, the inhibition of adsorption described herein is for reducing adhesion of pathogenic microorganisms (e.g., any biofilm-forming microorganism described herein which is potentially pathogenic) to a medical device.

In some embodiments of any of the embodiments described herein, adsorption of the biofouling-promoting agent (any biofouling-promoting agent described herein) on the surface of the substrate subjected to a method described herein (according to any of the respective embodiments) is reduced by at least 10% relative to adsorption on the surface of the substrate in the absence of the composition which comprises liposomes In some embodiments, adsorption is reduced by at least 20%. In some embodiments, adsorption is reduced by at least 30%. In some embodiments, adsorption is reduced by at least 40%. In some embodiments, adsorption is reduced by at least 50%. In some embodiments, adsorption is reduced by at least 60%. In some embodiments, adsorption is reduced by at least 70%. In some embodiments, adsorption is reduced by at least 80%. In some embodiments, adsorption is reduced by at least 90%.

Reduction of an amount of adsorbed biofouling-promoting agent may optionally be determined using any technique known in the art for detecting and quantifying an amount of agent, including, without limitation, using a labeled biofouling-promoting agent (e.g., as exemplified herein in the Examples section). The reduction is optionally measured by contacting each of the aforementioned surfaces (e.g., for 2 hours) with an aqueous solution (optionally comprising phosphate buffer, e.g., 0.1 M phosphate) of the biofouling-promoting agent (e.g., at 37° C. and/or pH 7), followed by repeated rinses to remove non-adsorbed agent (e.g., as exemplified in the examples section herein). The concentration of the biofouling-promoting agent in the aqueous solution is optionally 1 μg/ml or the concentration of a saturated solution of the agent, whichever concentration is lower.

According to an aspect of some embodiments of the invention, there is provided a composition comprising liposomes (according to any of the respective embodiments described herein), identified for use in inhibiting adsorption of a biofouling-promoting agent on a surface of a substrate (e.g., in accordance with any embodiment of a respective method described herein).

In some embodiments of any of the embodiments described herein, according to any of the aspects described herein, the composition comprising liposomes comprises a hydrogel (according to any of the respective embodiments described herein).

In some embodiments of any of the embodiments described herein, according to any of the aspects described herein, the composition comprising liposomes is a liquid comprising the liposomes. In some embodiments the liquid is an aqueous liquid (e.g., liposomes in water or in an aqueous solution).

In some embodiments of any of the embodiments described herein, contacting the substrate with a composition comprising liposomes is effected by applying a composition comprising a hydrogel (according to any of the respective embodiments described herein) and/or a composition which is a liquid (according to any of the respective embodiments described herein) onto a surface of the substrate.

In some embodiments, applying a composition onto a surface of the substrate is effected by rinsing and/or immersing at least a portion of the substrate in the composition. In some such embodiments, the composition is a liquid (according to any of the respective embodiments described herein).

Herein throughout, the term “rinsing” generally refers to brief contact (e.g., for several seconds) with a liquid, whereas the term “immersing” generally refers to longer periods of contact with a liquid. However, both terms refer to contact with a liquid, and are typically used herein together to encompass all forms of contact with a liquid, for example, in embodiments wherein the difference between rinsing and immersing is of no particular significance.

According to an aspect of some embodiments of the invention, there is provided an article comprising a substrate (as defined herein) and the composition identified for use in inhibiting adsorption of a biofouling-promoting agent (according to any of the respective embodiments described herein), the composition being applied on a surface of the substrate.

In some embodiments of any of the embodiments described herein, the article consists essentially of the substrate and the composition applied on a surface thereof.

In some embodiments of any of the embodiments described herein, the substrate and the composition applied on a surface thereof form only a portion of the article.

In some embodiments of any of the embodiments described herein, the substrate and the composition applied on a surface thereof form a structural component of the article. In some embodiments, the substrate and the composition applied on a surface thereof form only a portion of the article. In some embodiments, the substrate and the composition applied on a surface thereof are manufactured as a unit which is assembled with other units to form the article.

In some embodiments of any of the embodiments described herein, the article is a medical device (the substrate being a medical device or portion of a medical device, as described herein). In some embodiments, the medical device is a device designed to come into contact with a part of the body susceptible to infection, such as an internal portion of the body, a mucous membrane and/or a surface of the eye. Examples of such medical devices include, without limitation, surgical tools and implants (which are for coming into contact with an internal portion of the body) and contact lenses (which are for contacting a surface of the eye).

Exemplary articles include the following:

Medical devices such as, but are not limited to, pacemakers, heart valves, replacement joints, catheters, catheter access ports, dialysis tubing, gastric bands, shunts, screw plates, artificial spinal disc replacements, internal implantable defibrillators, cardiac resynchronization therapy devices, implantable cardiac monitors, mitral valve ring repair devices, left ventricular assist devices (LVADs), artificial hearts, implantable infusion pumps, implantable insulin pumps, stents, implantable neurostimulators, maxillofacial implants, dental implants, and the like;

Packages or containers, for example, food packages and containers, beverage packages and containers, medical device packages, agricultural packages and containers (of agrochemicals), blood sample or other biological sample packages and containers, and any other packages or containers of various articles;

Food packages such as packages of dairy products and/or containers for storage or transportation of dairy products;

Milk storage and processing devices such as, but not limited to, containers, storage tanks, raw milk holding equipments, dairy processing operations conveyer belts, tube walls, gaskets, rubber seals, stainless steel coupons, piping systems, filling machine, silo tanks, heat exchangers, post-pasteurization equipment, pumps, valves, separators, and spray devices;

Energy harvesting device, for example, a microelectronic device, a microelectromechanical device, a photovoltaic device and the like;

Microfluidic devices, for example, micropumps or micro valves and the like;

Sealing parts, for example, O rings, and the like;

Articles having a corrodible surface;

Agricultural devices, as, for example, described herein;

Textiles, for example, tough cottons;

Fuel transportation devices;

Construction elements, such as, but not limited to, paints, walls, windows, door handles, and the like;

Elements of water treatment systems (such as for containing and/or transporting and/or treating aqueous media or water), devices, containers, filters, tubes, solutions and gases and the likes; and

Elements of organic waste treatment systems (such as for containing and/or disposing and/or transporting and/or treating organic waste), devices, containers, filters, tubes, solutions and gases and the likes.

In some embodiments of any of the embodiments described herein, the article is characterized in that upon contact with the agent under biofouling-promoting conditions, a biofilm load on the substrate is lowered by at least 10% compared to a biofilm load on the substrate when being devoid of the composition (upon contact with the same agent under the same conditions). In some embodiments, the biofilm load is reduced by at least 20%. In some embodiments, the biofilm load is reduced by at least 30%. In some embodiments, the biofilm load is reduced by at least 40%. In some embodiments, the biofilm load is reduced by at least 50%. In some embodiments, the biofilm load is reduced by at least 60%. In some embodiments, the biofilm load is reduced by at least 70%. In some embodiments, the biofilm load is reduced by at least 80%. In some embodiments, the biofilm load is reduced by at least 90%.

Herein throughout, the term “biofilm-promoting conditions” refers to conditions suitable for formation and growth of a biofilm of a cell (e.g., P. aeruginosa), for example, wherein a surface is in contact (e.g., over the course of 1, 2, 3, 4, 5, 6 or 7 days) with an aqueous liquid (e.g., water or broth, optionally at 37° C.) containing such cells.

In some embodiments of any of the embodiments described herein, biofilm load is defined as an area of the biofilm.

In some embodiments of any of the embodiments described herein, biofilm load is defined as a mass and/or volume of the biofilm.

In some embodiments of any of the embodiments described herein, biofilm load is defined as a number of cells in the biofilm.

The biofilm load may optionally be determined using any technique known in the art for detecting and quantifying an amount of cells and/or microorganisms in a biofilm.

In some embodiments of any of the embodiments described herein, the time period of biofilm formation, after which biofilm load is determined, is in determined in accordance with the biofilm load, for example, the time period being a time period after which a biofilm covers 100%, 50%, or any other pre-determined percentage of the area of the substrate in the absence of inhibition of biofilm formation by contact with a composition comprising liposomes. For example, if a biofilm grows to cover 50% of a surface in the absence of biofilm formation inhibition, and during the same a time period, a biofilm grows to cover 30% of a surface in the presence of biofilm formation inhibition, then inhibition of biofilm formation may be considered to result in a reduction of 40% (i.e., (50%-30%)/50%) in biofilm formation.

Herein, the phrase “upon contact with the agent” means that in addition to the biofilm-promoting conditions, the agent is also present (e.g., in the aqueous liquid containing the cells).

Inhibition of Biofilm Formation:

According to an aspect of some embodiments of the invention, there is provided a method of inhibiting biofilm formation on a surface of a substrate (as defined herein in any embodiment and any combination of embodiments), the method comprising contacting (as described in any of the respective embodiments described herein) the substrate with a composition which comprises liposomes.

In some embodiments, “inhibiting biofilm formation” refers to the prevention of formation of a biofilm; and/or to a reduction in the rate of buildup of a biofilm; and/or to a reduction in the mass of a biofilm, the area or the volume of the biofilm, or in the number of cells forming the biofilm.

In some embodiments of any of the embodiments described herein, the inhibition of adsorption described herein is for reducing adhesion of pathogenic microorganisms (e.g., any biofilm-forming microorganism described herein which is potentially pathogenic) to a medical device. Such a reduction may result in inhibiting biofilm formation, as defined in some embodiments herein.

In some embodiments of any of the embodiments described herein, biofilm formation on the surface of the substrate subjected to a method described herein (according to any of the respective embodiments) is reduced by at least 10% relative to biofilm formation on the surface of the substrate in the absence of the composition which comprises liposomes. In some embodiments, biofilm formation is reduced by at least 20%. In some embodiments, biofilm formation is reduced by at least 30%. In some embodiments, biofilm formation is reduced by at least 40%. In some embodiments, biofilm formation is reduced by at least 50%. In some embodiments, biofilm formation is reduced by at least 60%. In some embodiments, biofilm formation is reduced by at least 70%. In some embodiments, biofilm formation is reduced by at least 80%. In some embodiments, biofilm formation is reduced by at least 90%.

The reduction in biofilm formation is optionally determined by measuring a biofilm load (in accordance with any of the respective embodiments described herein) for a biofilm of a cell (e.g., P. aeruginosa) on each surface after being subjected to biofouling-promoting conditions, as defined herein (e.g., over the course of 1, 2, 3, 4, 5, 6 or 7 days, or any other time period as described herein).

According to an aspect of some embodiments of the invention, there is provided a composition comprising liposomes (according to any of the respective embodiments described herein), identified for use in inhibiting biofilm formation on a surface of a substrate (e.g., in accordance with any embodiment of a respective method described herein).

In some embodiments of any of the embodiments described herein, according to any of the aspects described herein, the composition comprising liposomes comprises a hydrogel (according to any of the respective embodiments described herein).

In some embodiments of any of the embodiments described herein, according to any of the aspects described herein, the composition comprising liposomes is a liquid comprising the liposomes (according to any of the respective embodiments described herein). In some embodiments the liquid is an aqueous liquid (e.g., liposomes in water or in an aqueous solution), according to any of the respective embodiments described herein.

In some embodiments of any of the embodiments described herein, contacting the substrate with a composition comprising liposomes is effected by applying a composition comprising a hydrogel (according to any of the respective embodiments described herein) and/or a composition which is a liquid (according to any of the respective embodiments described herein) onto a surface of the substrate.

In some embodiments, applying a composition onto a surface of the substrate is effected by rinsing and/or immersing at least a portion of the substrate in the composition. In some such embodiments, the composition is a liquid (according to any of the respective embodiments described herein).

According to an aspect of some embodiments of the invention, there is provided an article comprising a substrate (as defined herein) and the composition identified for use in inhibiting biofilm formation (according to any of the respective embodiments described herein), the composition being applied on a surface of the substrate.

In some embodiments of any of the embodiments described herein, the article consists essentially of the substrate and the composition applied on a surface thereof.

In some embodiments of any of the embodiments described herein, the substrate and the composition applied on a surface thereof form a structural component of the article. In some embodiments, the substrate and the composition applied on a surface thereof are manufactured as a unit which is assembled with other units to form the article.

In some embodiments of any of the embodiments described herein, the article is a medical device. In some embodiments, the medical device is a device designed to come into contact with a part of the body susceptible to infection, such as an internal portion of the body, a mucous membrane and/or a surface of the eye. Examples of such medical devices include, without limitation, surgical tools, implants and contact lenses.

In some embodiments of any of the embodiments described herein, the article is characterized in that under biofouling-promoting conditions, a biofilm load on the substrate is lowered by at least 10% compared to a biofilm load on the substrate when being devoid of the composition (under the same conditions). In some embodiments, the biofilm load is reduced by at least 20%. In some embodiments, the biofilm load is reduced by at least 30%. In some embodiments, the biofilm load is reduced by at least 40%. In some embodiments, the biofilm load is reduced by at least 50%. In some embodiments, the biofilm load is reduced by at least 60%. In some embodiments, the biofilm load is reduced by at least 70%. In some embodiments, the biofilm load is reduced by at least 80%. In some embodiments, the biofilm load is reduced by at least 90%.

Hydrogel and Liposomes in Composition:

According to an aspect of some embodiments of the invention, there is provided a method of inhibiting adsorption of a biofouling-promoting agent on a surface of a substrate (in accordance with any of the respective embodiments described herein) and/or a method of inhibiting biofilm formation on a surface of a substrate (in accordance with any of the respective embodiments described herein), the method comprising contacting the substrate with a composition which comprises liposomes and a hydrogel.

According to an aspect of some embodiments of the invention, there is provided a composition which comprises liposomes and a hydrogel.

In some embodiments, the composition is identified for use in inhibiting adsorption of a biofouling-promoting agent on a surface of a substrate (in accordance with any of the respective embodiments described herein).

In some embodiments, the composition is identified for use in inhibiting biofilm formation on a surface of a substrate (in accordance with any of the respective embodiments described herein).

In some embodiments of any one of the embodiments described herein, according to any of the aspects described herein, the hydrogel comprises liposomes. In some embodiments, the hydrogel comprises substantially all (e.g. at least 90%, at least 95%, at least 98%, at least 99%) of the liposomes in the composition.

In some embodiments of any one of the embodiments described herein, according to any of the aspects described herein, the composition is in a form of a hydrogel comprising the liposomes.

In some embodiments of any one of the embodiments described herein, inhibiting adsorption and/or biofilm formation is effected by applying the composition comprising a hydrogel onto a surface of the substrate, thereby contacting the substrate with the composition. Such a substrate may be any type of substrate described herein.

In some embodiments of any one of the embodiments described herein, the substrate is not composed of a hydrogel. In some embodiments, the substrate does not comprise a hydrogel.

Without being bound by any particular theory, it is believed that substrates which are not composed of a hydrogel, and particularly substrates which do not comprise a hydrogel, are particularly suitable for being treated with a composition comprising a hydrogel and liposomes as described herein, as they cannot be contacted with liposomes by incorporating liposomes within a hydrogel in the substrate, as described herein.

In some embodiments of any one of the embodiments described herein, the composition is in a malleable (e.g., in a form of a viscous fluid), which may optionally be applied onto a surface, for example, by being spread onto the surface. In some such embodiments, the hydrogel is malleable. In some such embodiments, the composition comprises a plurality of hydrogel particles, and the composition is malleable due to the ability of the hydrogel particles to move with respect to one another.

In some embodiments of any one of the embodiments described herein, the composition is applied onto a surface by being formed (e.g., in accordance with any of the respective embodiments described herein) on the surface. In some such embodiments, the composition is not malleable.

In some embodiments of any one of the embodiments described herein, a composition comprising a hydrogel (according to any of the respective embodiments described herein) is prepared (e.g., as part of a method described herein) by forming the hydrogel in the presence of liposomes.

In some embodiments of any one of the embodiments described herein, forming a hydrogel comprises, for example, contacting an aqueous liquid with a polymer which forms a hydrogel upon contact with water; and/or forming, in an aqueous liquid, a polymer which forms a hydrogel upon contact with water (e.g., by cross-linking a polymer which does not form a hydrogel prior to cross-linking, to thereby form a cross-linked polymer which forms a hydrogel). In some embodiments, the aqueous liquid comprises liposomes, thereby forming a hydrogel in the presence of liposomes.

In some embodiments of any one of the embodiments described herein, the liposomes are dispersed throughout the bulk of the hydrogel.

In some embodiments of any one of the embodiments described herein, forming the hydrogel is such that the liposomes, and optionally any other component of the hydrogel described herein, are dispersed throughout the bulk of the hydrogel. In some embodiments, dispersion throughout the bulk of a hydrogel is effected by cross-linking a polymer in a substantially homogeneous aqueous liquid comprising the liposomes (and optionally other hydrogel components described herein), to thereby form a cross-linked polymer which forms a hydrogel.

Examples of procedures for forming a hydrogel in the presence of liposomes, which may optionally be used to form a hydrogel contacted with liposomes according to any of the respective embodiments described herein, are presented in the Examples section herein below, as well as in International Patent Application PCT/IL2014/050604 (the contents of which are incorporated herein by reference in their entirety).

In some embodiments of any one of the embodiments described herein, the hydrogel is such that inhibition of adsorption (quantified as a percent in reduction of adsorbed agent) is substantially unchanged after a dehydration-rehydration cycle of the hydrogel, that is, dehydration of the hydrogel (e.g., by lyophilization) followed by rehydration of the hydrogel to the water content prior to the dehydration. In some embodiments, inhibition of adsorption is substantially unchanged even after more than one dehydration-rehydration cycle of the hydrogel, for example, at least 2, 3, 4, 5, and even 10 cycles.

In some embodiments of any one of the embodiments described herein, the hydrogel is such that inhibition of biofilm formation (quantified as a percent in reduction of a biofilm size) is substantially unchanged after a dehydration-rehydration cycle of the hydrogel. In some embodiments, inhibition of biofilm formation is substantially unchanged even after more than one dehydration-rehydration cycle of the hydrogel, for example, at least 2, 3, 4, 5, and even 10 cycles.

As used herein, the phrase “substantially unchanged” refers to a value within a range of 50% to 150% of an original value. For example, if inhibition of adsorption prior to dehydration-rehydration is 50% (that is, the inhibition effects a 50% reduction in adsorption relative to a substrate not contacted with a composition comprising liposomes), and inhibition of adsorption subsequent to dehydration-rehydration is 40% (that is, the inhibition effects a 40% reduction in adsorption), the inhibition subsequent to dehydration-rehydration is 80% of the original inhibition (i.e., 40% divided by 50%), and is therefore considered herein to be substantially unchanged.

In some embodiments of any one of the embodiments described herein, a substantially unchanged value described herein (e.g., inhibition of adsorption and/or biofilm formation) is in a range of 70% to 130% of an original value. In some embodiments, a substantially unchanged value is in a range of 80% to 120% of an original value. In some embodiments, a substantially unchanged value is in a range of 90% to 110% of an original value.

Hydrogel Substrate with Liposomes:

According to an aspect of some embodiments of the invention, there is provided a method of inhibiting adsorption of a biofouling-promoting agent on a surface of a substrate (in accordance with any of the respective embodiments described herein) and/or a method of inhibiting biofilm formation on a surface of a substrate (in accordance with any of the respective embodiments described herein), the method comprising contacting a substrate composed of a hydrogel with a composition which comprises liposomes.

Herein, the phrase “composed of a hydrogel” refers to a substance (e.g., a substrate described herein) in which at least a portion of the substance consists of a hydrogel. In some of any of the respective embodiments, the substance composed of a hydrogel (e.g., a substrate composed of a hydrogel as described herein) primarily consists of a hydrogel, that is, more than 50% of the volume of the substrate is a hydrogel. In some embodiments of any one of the embodiments described herein, the substrate consists essentially of a hydrogel.

In some embodiments of any one of the embodiments described herein, at least 50% of an area of a surface of the substrate (for which adsorption and/or biofilm formation is being inhibited according to any of the embodiments described herein) is a surface of a hydrogel. In some embodiments, at least 60% of an area of the surface of the substrate is a surface of a hydrogel. In some embodiments, at least 70% of an area of the surface of the substrate is a surface of a hydrogel. In some embodiments, at least 80% of an area of the surface of the substrate is a surface of a hydrogel. In some embodiments, at least 90% of an area of the surface of the substrate is a surface of a hydrogel. In some embodiments, at least 99% of an area of the surface of the substrate is a surface of a hydrogel.

In some embodiments of any one of the embodiments described herein, the contacting of the substrate with the composition comprising liposomes comprises incorporating the composition within the hydrogel. In some such embodiments, the composition consists essentially of the liposomes. In some such embodiments, the composition consists essentially of the liposomes and a liquid carrier (e.g., water or an aqueous solution).

In some embodiments of any one of the embodiments described herein, the substrate is prepared (e.g., as part of a method described herein) by forming a hydrogel which comprises liposomes incorporated in and/or on the hydrogel, thereby contacting the substrate with the liposomes.

In some embodiments of any one of the embodiments described herein, the substrate composed of a hydrogel (according to any of the respective embodiments described herein) is prepared (e.g., as part of a method described herein) by forming the hydrogel in the presence of liposomes.

In some embodiments of any one of the embodiments described herein, forming a hydrogel comprises, for example, contacting an aqueous liquid with a polymer which forms a hydrogel upon contact with water; and/or forming, in an aqueous liquid, a polymer which forms a hydrogel upon contact with water (e.g., by cross-linking a polymer which does not form a hydrogel prior to cross-linking, to thereby form a cross-linked polymer which forms a hydrogel). In some embodiments, the aqueous liquid comprises liposomes, thereby forming a hydrogel in the presence of liposomes.

In some embodiments of any one of the embodiments described herein, the liposomes are dispersed throughout the bulk of the hydrogel.

In some embodiments of any one of the embodiments described herein, forming the hydrogel is such that the liposomes, and optionally any other component of the hydrogel described herein, are dispersed throughout the bulk of the hydrogel. In some embodiments, dispersion throughout the bulk of a hydrogel is effected by cross-linking a polymer in a substantially homogeneous aqueous liquid comprising the liposomes (and optionally other hydrogel components described herein), to thereby form a cross-linked polymer which forms a hydrogel.

Examples of procedures for forming a hydrogel in the presence of liposomes, which may optionally be used to form a hydrogel contacted with liposomes according to any of the respective embodiments described herein, are presented in the Examples section herein below, as well as in International Patent Application PCT/IL2014/050604 (the contents of which are incorporated herein by reference in their entirety).

In some embodiments of any one of the embodiments described herein, the substrate is prepared by forming the hydrogel of the substrate in the presence of a solution of polymer (according to any of the respective embodiments described herein), optionally in addition to the liposomes. In some embodiments, the polymer is not a polymer which forms a hydrogel, but rather an additive, for example, a polymer (e.g., a hydrophilic and/or non-cross-linked polymer described herein) which enhances inhibition of adsorption and/or biofilm formation (e.g., as described herein).

In some embodiments of any one of the embodiments described herein, a polymer (which is not a polymer which forms a hydrogel) is dispersed throughout the bulk of the hydrogel. In some embodiments, the polymer which forms a hydrogel is intermixed with a polymer (e.g., a cross-linked polymer) which forms the hydrogel.

In some embodiments of any one of the embodiments described herein, forming the hydrogel is such that a polymer (which is not a polymer which forms a hydrogel) is dispersed throughout the bulk of the hydrogel. In some embodiments, dispersion throughout the bulk of a hydrogel is effected by cross-linking a polymer (which forms a polymer upon cross-linking), to thereby form a cross-linked polymer which forms a hydrogel, in a substantially homogeneous aqueous liquid comprising the polymer which does not form a hydrogel (e.g., a solution of the polymer which does not form a hydrogel), to thereby provide polymer dispersed throughout the bulk of the hydrogel.

Liposomes and Lipids:

The liposomes and/or lipids according to any one of the embodiments described in this section may be used in the context of any one of the embodiments of any of the aspects of the inventions described herein.

As used herein and in the art, the term “liposome” refers to an artificially prepared vesicle comprising a bilayer composed of molecules of an amphiphilic lipid. In an aqueous medium, the bilayer is typically configured such that hydrophilic moieties of the amphiphilic lipid are exposed to the medium at both surfaces of the bilayer, whereas lipophilic moieties of the lipid are located in the internal portion of the bilayer, and therefore less exposed to the medium. Examples of liposomes which may be used in any one of the embodiments described herein include, without limitation, small unilamellar vesicles, large unilamellar vesicles and multilamellar vesicles.

In some embodiments of any one of the embodiments described herein, the liposomes comprise multilamellar vesicles. In some embodiments, the liposomes are primarily (more than 50 weight percents) multilamellar vesicles.

In some embodiments of any one of the embodiments described herein, the liposomes comprise small unilamellar vesicles. In some embodiments, the liposomes are primarily (more than 50 weight percents) small unilamellar vesicles.

In some embodiments of any one of the embodiments described herein, the liposomes comprise large unilamellar vesicles. In some embodiments, the liposomes are primarily (more than 50 weight percents) large unilamellar vesicles.

As used herein, the term “unilamellar” refers to liposomes characterized by a single lipid bilayer, whereas the term “multilamellar” refers to liposomes characterized by a multiple lipid bilayers, for example, concentric bilayers.

As used herein, the phrase “small unilamellar vesicle” refers to unilamellar liposomes of less than 100 nm in diameter, whereas the phrase “large unilamellar vesicle” refers to unilamellar liposomes at least 100 nm in diameter.

As used herein, the term “amphiphilic lipid” refers to compounds comprising at least one hydrophilic moiety and at least one lipophilic moiety. Examples of amphiphilic lipids include, without limitation, fatty acids (e.g., at least 6 carbon atoms in length) and derivatives thereof such as phospholipids and glycolipids; sterols (e.g., cholesterol) and steroid acids.

In some embodiments of any one of the embodiments described herein, contacting a substrate with the composition comprising liposomes comprises coating the surface with an amphiphilic lipid of the liposomes. Optionally, the surface is coated by a layer of liposomes.

Upon contact with substrate, amphiphilic lipids in a form of liposomes may optionally rearrange in a form other than a liposome, such as a layer (e.g., lipid monolayer and/or lipid bilayer) coating a least a portion of a surface of the substrate.

As used herein, the phrases “amphiphilic lipid of the liposomes”, “amphiphilic lipid of said liposomes” and the like refer to any amphiphilic lipid which is present in the liposomes according to any of the respective embodiments described herein, and encompasses such lipids even if they are no longer in a liposome (e.g., if the liposome is destroyed).

Herein, the term “phospholipid” refers to a compound comprising a substituted or non-substituted phosphate group and at least one alkyl chain (optionally at least two alkyl chains) which is optionally at least 5 carbon atoms in length, optionally at least 7 atoms in length and optionally at least 9 atoms in length. The at least one alkyl chain is optionally a part of an acyl group (e.g., a fatty acid residue) or an alkyl group per se (e.g., a fatty alcohol residue). In some embodiments, the phosphate group and on e or two (optionally two) alkyl chains (e.g., acyl or alkyl) are attached to a glycerol moiety via the oxygen atoms of glycerol. The term “phospholipid” encompasses lipids having a (phosphorylated) glycerol backbone (e.g., monoacylglyceride and/or diacylglyceride phospholipids), referred to as glycerophospholipids; and lipids having a (phosphorylated) sphingosine backbone, referred to as phosphosphingolipids (e.g., sphingomyelins).

As used herein, the term “glycolipid” encompasses lipids having a (glycosylated) glycerol backbone (e.g., monoacylglyceride and/or diacylglyceride glycolipids), referred to as glyceroglycolipids; and lipids having a (glycosylated) sphingosine backbone, referred to as glycosphingolipids (e.g., cerebrosides, gangliosides).

In some embodiments of any one of the embodiments described herein, the amphiphilic lipid comprises a hydrophilic moiety which is an ionic moiety.

Herein, the phrase “ionic moiety” refers to a moiety which comprises at least one charged group (as defined herein), and includes anionic moieties (which have a net negative charge), cationic moieties (which have a net positive charge) and zwitterionic moieties (which have an equal number of positive and negative charges, and thus, no net charge).

Without being bound by any particular theory, it is believed that ionic moieties are particularly effective at binding to water molecules, which renders lipid molecules comprising such moieties particularly effective at promoting hydration layers near the surface which can serve as a barrier to adsorption onto the surface.

In some embodiments of any one of the embodiments described herein, the amphiphilic lipid of the liposomes comprises at least one phospholipid. Phospholipids are typically characterized by the presence of an ionic moiety which includes a negative charge associated with an oxygen atom in a phosphate moiety (P—O⁻), although additional charges may be present.

In some embodiments of any one of the embodiments described herein, the phospholipid is a glycerophospholipid. In some embodiments, the glycerophospholipid is a diacylglyceride, comprising two fatty acyl groups and one phosphate group attached to a glycerol backbone.

In some embodiments of any one of the embodiments described herein, a concentration of phospholipids in liposomes in a composition described herein (e.g., a solution described herein) is in a range of from 0.1 mM to 500 mM. In some embodiments, the concentration is in a range of from 0.3 mM to 150 mM. In some embodiments, the concentration is in a range of from 1 mM to 50 mM. In some embodiments, the concentration is in a range of from 2 mM to 15 mM.

In some embodiments of any one of the embodiments described herein, a concentration of phospholipids in liposomes in a composition described herein (e.g., a solution described herein) is in a range of from 0.1 mM to 50 mM. In some embodiments, the concentration is in a range of from 0.3 mM to 15 mM.

In some embodiments of any one of the embodiments described herein, a concentration of phospholipids in liposomes in a composition described herein (e.g., a solution described herein) is in a range of from 0.3 mM to 500 mM. In some embodiments, the concentration is in a range of from 1 mM to 150 mM. In some embodiments, the concentration is in a range of from 2 mM to 50 mM.

In some embodiments of any one of the embodiments described herein, the amphiphilic lipid of the liposomes comprises at least one negatively charged atom and at least one positively charged atom. In some embodiments, the amphiphilic lipid is zwitterionic, that is, the one or more negative charges in the molecule are balanced out by an equal number of positive charge(s) in the molecule. In some embodiments, the amphiphilic lipid comprises exactly one negative charge and one positive charge.

In some embodiments of any one of the embodiments described herein, the amphiphilic lipid of the liposomes comprises at least one phospholipid which comprises a phosphoethanolamine group or N-alkyl derivative thereof. In some embodiments, the phospholipid is a glycerophospholipid.

The phrase “phosphoethanolamine group or N-alkyl derivative thereof” refers to a —O—P(═O)(—O⁻)—OCH₂CH₂NR′R″R′″⁺ group (or a salt thereof), wherein R′, R″ and R′″ are each independently hydrogen or alkyl, preferably C₁₋₄ alkyl. In some embodiments of any one of the embodiments described herein, the alkyl groups attached to the nitrogen atom are each independently methyl or ethyl. In some embodiments, the alkyl(s) is methyl. The term “phosphoethanolamine” refers to a group wherein R′, R″ and R′″ are each hydrogen. The term “phosphocholine” refers to a group wherein R′, R″ and R′″ are each methyl.

Without being bound by any particular theory, it is believed that the distance between the positive and negative charges in a phosphoethanolamine group or N-alkyl derivative thereof is particularly suitable for binding water molecules and/or promoting hydration.

In some embodiments of any one of the embodiments described herein, a molar percentage of the phospholipid described herein (e.g., in liposomes described herein) which comprises a phosphoethanolamine group or N-alkyl derivative thereof is at least 20%. In some embodiments, the molar percentage is at least 40%. In some embodiments, the molar percentage is at least 50%. In some embodiments, the molar percentage is at least 60%. In some embodiments, the molar percentage is at least 70%. In some embodiments, the molar percentage is at least 80%. In some embodiments, the molar percentage is at least 90%. In some embodiments, the phospholipid consists essentially of at least one phospholipid comprising a phosphoethanolamine group or N-alkyl derivative thereof.

In some embodiments of any one of the embodiments described herein, a molar percentage of the amphiphilic lipid of the liposomes described herein which consists of at least one phospholipid which comprises a phosphoethanolamine group or N-alkyl derivative thereof is at least 20%. In some embodiments, the molar percentage is at least 40%. In some embodiments, the molar percentage is at least 50%. In some embodiments, the molar percentage is at least 60%. In some embodiments, the molar percentage is at least 70%. In some embodiments, the molar percentage is at least 80%. In some embodiments, the molar percentage is at least 90%. In some embodiments, the amphiphilic lipid consists essentially of at least one phospholipid which comprises a phosphoethanolamine group or N-alkyl derivative thereof.

In some embodiments of any one of the embodiments described herein, the at least one phospholipid comprises at least one phosphatidylcholine.

Herein and in the art, the term “phosphatidylcholine” refers to a glycerophospholipid comprising a phosphocholine group and two fatty acyl groups attached to a glycerol backbone (i.e., a diacylglyceride).

In some embodiments of any one of the embodiments described herein, the phospholipid described herein (e.g., in liposomes described herein) is characterized by a molar percentage of phosphatidylcholine (the at least one phosphatidylcholine described herein) which is at least 20%. In some embodiments, the molar percentage is at least 40%. In some embodiments, the molar percentage is at least 50%. In some embodiments, the molar percentage is at least 60%. In some embodiments, the molar percentage is at least 70%. In some embodiments, the molar percentage is at least 80%. In some embodiments, the molar percentage is at least 90%. In some embodiments, the phospholipid consists essentially of at least one phosphatidylcholine.

In some embodiments of any one of the embodiments described herein, the amphiphilic lipid of the liposomes described herein is characterized by a molar percentage of phosphatidylcholine (the at least one phosphatidylcholine described herein) which is at least 20%. In some embodiments, the molar percentage is at least 40%. In some embodiments, the molar percentage is at least 50%. In some embodiments, the molar percentage is at least 60%. In some embodiments, the molar percentage is at least 70%. In some embodiments, the molar percentage is at least 80%. In some embodiments, the molar percentage is at least 90%. In some embodiments, the amphiphilic lipid consists essentially of at least one phosphatidylcholine.

The fatty acyl groups in a lipid described herein may comprise saturated fatty acyl groups, monounsaturated fatty acyl groups (having a single unsaturated bond) and/or polyunsaturated fatty acyl groups (having two or more unsaturated bonds). In some embodiments, the unsaturated bonds are cis double bonds.

Examples of suitable saturated fatty acyl groups include, without limitation, lauroyl, myristoyl, palmitoyl and stearoyl.

Examples of suitable monounsaturated fatty acyl groups include, without limitation, oleoyl, palmitoleoyl, eicosenoyl, erucoyl, nervonoyl and vaccenoyl.

Examples of suitable polyunsaturated fatty acyl groups include, without limitation, linoleoyl, α-linolenoyl, γ-linolenoyl, dihomo-γ-linolenoyl, stearidonoyl, eicosatetraenoyl, eicosapentaenoyl, docosapentaenoyl, docosahexaenoyl, arachidonoyl and adrenoyl.

In some embodiments of any one of the embodiments described herein, the fatty acyl groups are selected from the group consisting of saturated and monounsaturated fatty acyl groups. In some embodiments, the fatty acyl groups are saturated fatty acyl groups.

Without being bound by any particular theory, it is believed that saturated and monounsaturated fatty acyl groups, particularly saturated fatty acyl groups, are relatively resistant to chemical reaction such as oxidation, and therefore provide a more resilient system.

In some embodiments of any one of the embodiments described herein, at least 50% of the fatty acyl groups are the same species of fatty acyl group (e.g., myristoyl, palmitoyl). In some embodiments, at least 75% of the fatty acyl groups are the same species of fatty acyl group. In some embodiments, at least 90% of the fatty acyl groups are the same species of fatty acyl group.

1,2-dimyristoyl-sn-glycero-3-phosphocholine is an example of a phospholipid comprising a single species of fatty acyl group.

It is to be appreciated that phase transitions, e.g., melting points (Tm), of the lipid bilayers and liposomes described herein may be determined by the skilled person by selecting suitable fatty acyl groups for inclusion in the lipids, for example, by selecting relatively short and/or unsaturated fatty acyl groups (e.g., myristoyl) to obtain a relatively low melting point; and/or by selecting relatively long and/or saturated fatty acyl groups (e.g., palmitoyl and/or stearoyl) to obtain a relatively high melting point.

In some embodiments of any one of the embodiments described herein, the liposomes described herein are characterized by a phase transition melting point below an expected ambient temperature of a surface to which the liposomes are applied (e.g., as described herein in any one of the respective embodiments), such that a surface coated by lipids at the expected ambient temperature (e.g., about 36° C. on a contact lens surface in contact with an eye, about 20° C. or 25° C. in a non-physiological environment) will be coated predominantly by lipids in a liquid phase.

In some embodiments of any one of the embodiments described herein, the liposomes are characterized by a phase transition melting point (Tm) below 37° C. In some embodiments, the Tm is below 30° C. In some embodiments, the Tm is below 25° C. In some embodiments, the Tm is below 20° C.

1,2-dimyristoyl-sn-glycero-3-phosphocholine (Tm=24° C.) is an example of a phospholipid characterized by a Tm below 25° C. Thus, liposomes consisting essentially of such a phospholipid will be characterized by a Tm of 24° C. Determination of a Tm of liposomes containing 1,2-dimyristoyl-sn-glycero-3-phosphocholine in combination with one or more other lipids is well withing the capabilities of one of skill in the art.

In some embodiments of any one of the embodiments relating to a method described herein, the liposomes described herein are characterized by a phase transition melting point below a temperature of a surface (e.g., a temperature described herein) during contacting of the liposomes with the surface (according to any of the respective embodiments described herein).

In some embodiments described herein, liquid phase liposomes exhibit a superior anti-biofouling effect as compared to solid phase liposomes.

Lipid coatings in a liquid phase have been reported previously to provide the most effective hydration and/or lubrication.

However, in some embodiments described herein wherein liquid phase liposomes are superior to solid phase liposomes in providing an anti-biofouling effect, such superiority is considerably greater than the differences in hydration and/or lubrication efficacy between liquid phase and solid phase liposomes, for example, due to a superior ability of liquid phase liposomes to coat a surface with a lipid with minimal gaps in a lipid layer on a surface.

In some embodiments of any one of the embodiments described herein wherein the liposomes are characterized by a relatively high phase transition melting point (Tm) (e.g., at least 37° C., at least 30° C., at least 25° C.) and/or wherein the liposomes described herein are characterized by a phase transition melting point above a temperature of a surface during contacting of the liposomes with the surface (according to any of the respective embodiments described herein), the liposomes are used in combination with a polymer according to any of the respective embodiments described herein. In some such embodiments, the polymer comprises hydrophobic regions therein (e.g., as described herein).

In some embodiments of any one of the embodiments described herein, the liposomes described herein are characterized by a surface charge, which may be a positive surface charge or a negative surface charge.

As used herein, the phrase “surface charge” refers to an electric charge at or near a surface, such as an interface of a liposome with a solution. The phrase “surface charge” encompasses an electric charge associated with an electric potential at a surface (e.g., such that a positive electric potential at a surface is indicative of a positive surface charge, whereas a negative electric potential at a surface is indicative of a negative surface charge); as well as an electric charge which is closer to a surface than an electric charge of an opposite sign (e.g., as in a zwitterion wherein the positive charge is closer to the surface than the negative charge, or vice versa), such that an ion near the surface will interact primarily with the electric charge near the surface (due to the proximity) as opposed to the electric charge of an opposite sign. For example, phosphatidylcholine liposomes typically exhibit a positive surface charge because the positive charge of the choline group is closer to the liposome surface than the negative charge of the phosphate group.

Optionally, a surface charge of a liposome is associated with a net charge of the lipid molecules in the liposome, for example, a liposome comprising anionic lipids has a negative surface charge, and/or a liposome comprising cationic lipids has a positive surface charge.

Alternatively or additionally, a surface charge of a liposome is associated with a dipole of lipid molecules (e.g., zwitterionic lipid molecules) in the liposome, for example, a liposome comprising a zwitterionic lipid comprising a phosphocholine group may have a positive surface charge due to the positively charged ammonium groups in the phosphocholine groups being (on average) closer to the surface of the liposomes than the negatively charged phosphate groups in the phosphocholine groups.

The skilled person will be readily capable of determining a surface charge. For example, the sign of a surface charge may be determined by comparing the propensity of a surface (e.g., of a liposome) to bind to anionic vs. cationic compounds (e.g., labeling compounds).

In some embodiments of any one of the embodiments described herein, the liposomes rupture upon contact with the substrate (e.g., in embodiments in which the liposomes are contacted with a surface of the substrate). Liposome rupture may optionally result in a lipid bilayer in the liposomes being converted from a curved geometry (e.g., as in the relatively spherical liposomes) to a flatter geometry which complements the geometry of the substrate surface and/or molecules attached to the surface (e.g., thereby enhancing affinity of the lipids to the surface), thereby facilitating coating of the surface with a lipid (e.g., as described herein); and/or which results in a flatter, smoother lipid-coated surface, which may further inhibit adsorption and/or biofilm formation on the surface.

Without being bound by any particular theory, it is believed that rupture of liposomes may be induced by affinity of the polymer described herein to the lipids in the liposome, whereby rupture of the liposomes allows a greater area of the polymer to come into contact with lipids, thereby increasing an amount of energetically favorable interactions between the polymer and lipid.

In some embodiments of any one of the embodiments described herein, liposomes and polymer are selected such that the selected polymer is effective at rupturing the selected liposomes.

Polymer:

In some embodiments of any of the embodiments of any of the aspects of the invention described herein, a polymer is utilized in combination with the liposomes described herein.

Herein throughout, the term “polymer” refers to a polymer utilized in combination with the liposomes (as opposed, for example, to a component of a substrate and/or hydrogel described herein), unless indicated otherwise.

The polymer according to any one of the embodiments described in this section may be used in the context of any one of the embodiments of any of the aspects of the inventions described herein, and in combination with liposomes according to any one of the embodiments described herein with respect to liposomes.

In some embodiments of any of the embodiments described herein relating to a method, the method further comprises contacting the substrate with the polymer.

In some embodiments of any of the embodiments described herein, contacting the substrate with the polymer is effected prior to contacting the substrate with the composition comprising liposomes.

In some embodiments of any of the embodiments described herein, contacting the substrate with the polymer is effected subsequent to contacting the substrate with the composition comprising liposomes.

In some embodiments of any of the embodiments described herein, contacting the substrate with the polymer is effected concomitantly with contacting the substrate with the composition comprising liposomes.

In some embodiments of any of the embodiments described herein, according to any of the aspects described herein, the composition comprising liposomes further comprises the polymer. Such a composition may optionally be used to effect concomitant contacting of the substrate with the composition and polymer.

In some embodiments of any of the embodiments described herein, the composition comprising the polymer and liposomes is a liquid (e.g., as described herein) and the polymer is dissolved and/or dispersed in the liquid. In some embodiments, the polymer is dissolved in the liquid.

In some embodiments of any of the embodiments described herein, the composition comprising the polymer and liposomes comprises a hydrogel (e.g., as described herein) and the polymer is dispersed throughout the bulk of the hydrogel.

In some embodiments of any of the embodiments described herein relating to a composition comprising a hydrogel, the composition is prepared by forming the hydrogel in the presence of a solution of the polymer. In some embodiments, the forming of the hydrogel is such that the polymer is dispersed throughout the bulk of the hydrogel.

In some embodiments of any of the embodiments described herein, the polymer utilized in combination with the liposomes is not a polymer which forms a hydrogel described herein.

In some embodiments of any of the embodiments described herein, the polymer is a non-cross-linked polymer. In some such embodiments, the hydrogel is formed from a cross-linked polymer, and the non-cross-linked polymer is thus different than the polymer which forms a hydrogel.

Herein, the phrase “non-cross-linked polymer” encompasses polymers in which a degree of cross-linking (e.g., covalent cross-linking) is sufficiently low such that the molecules of the polymer (which optionally comprise a plurality of cross-linked polymer chains) have an average molecular weight (weight-averaged average molecular weight) which is not more than 10 MDa (10,000,000 Da).

In some embodiments of any of the embodiments described herein, an average molecular weight (weight-averaged average molecular weight) of the polymer is 3 MDa (3,000,000 Da) or less. In some embodiments, the average molecular weight of the polymer is 1 MDa or less. In some embodiments, the average molecular weight of the polymer is 300 kDa or less. In some embodiments, the average molecular weight of the polymer is 100 kDa or less. In some embodiments, the average molecular weight of the polymer is 30 kDa or less.

In some embodiments of any of the embodiments described herein, the polymer is a hydrophilic polymer.

Herein, the phrase “hydrophilic polymer” encompasses polymers which are water-soluble and/or water-swellable.

In some embodiments of any of the embodiments described herein, the hydrophilic polymer is water-soluble.

In some embodiments of any of the embodiments described herein, the hydrophilic polymer is water-swellable.

Herein, the term “water-soluble” refers to a solubility of at least 1 gram per liter in water at a pH of 7.

In some embodiments of any of the embodiments described herein, a water-soluble polymer has a solubility of at least 3 grams per liter in water at a pH of 7. In some embodiments, the solubility is at least 10 grams per liter. In some embodiments, the solubility is at least 30 grams per liter. In some embodiments, the solubility is at least 100 grams per liter.

Herein, the term “water-swellable” refers to an ability of a solid substance to absorb at least 10 weight percents water (weight of water per weight of solid) upon contact with water at a pH of 7.

In some embodiments of any of the embodiments described herein, a water-swellable polymer has an ability to absorb at least 30 weight percents water (weight of water per weight of solid) upon contact with water at a pH of 7. In some embodiments, the water-swellable polymer has an ability to absorb at least 20 weight percents water. In some embodiments, the water-swellable polymer has an ability to absorb at least 50 weight percents water. In some embodiments, the water-swellable polymer has an ability to absorb at least 100 weight percents water. In some embodiments, the water-swellable polymer has an ability to absorb at least 200 weight percents water (twice the weight of the polymer per se).

Examples of hydrophilic polymers include, without limitation, polypeptides, such as gelatin; non-ionic hydrophilic polymers, such as poly(2-hydroxyethyl methacrylate), hydroxypropyl methyl cellulose (HPMC), hydroxypropyl cellulose (HPC), methyl cellulose, dextran, polyvinylpyrrolidone (PVP), polyethylene glycol (also referred to herein interchangeably as “polyethylene oxide” or “PEO”) and polyvinyl alcohol; ionic polymers, such as polymethacrylic acid, carboxymethyl cellulose, hyaluronic acid, chondroitin sulfate, alginic acid, xanthan gum, chitosan and N-alkyl chitosan derivatives; and copolymers thereof. Poly(2-hydroxyethyl methacrylate), polyvinylpyrrolidone, polyethylene oxide and hydroxypropyl methyl cellulose are exemplary non-ionic hydrophilic polymers.

In some embodiments of any of the embodiments described herein relating to a polymer, the polymer is a non-ionic polymer, for example, a polymer selected from the group consisting of polyvinylpyrrolidone, polyethylene oxide and hydroxypropyl methyl cellulose.

In some embodiments of any of the embodiments described herein relating to a polymer, the polymer is hydroxypropyl methyl cellulose. As exemplified herein, both solid phase and liquid phase liposomes in combination with hydroxypropyl methyl cellulose are effective at reducing adhesion.

In some embodiments of any of the embodiments described herein, the polymer comprises hydrophobic regions (e.g., in embodiments wherein the liposomes have a relatively high melting point and/or have a melting point above a temperature of a surface to which they are contacted, as described herein).

Examples of polymers (e.g., hydrophilic polymers) comprising hydrophobic regions include, without limitation, cellulose derivatives (e.g., HPMC, HPC, methyl cellulose, ethyl cellulose, carboxymethyl cellulose), poly(vinyl methyl ether), polyethacrylic acid and esters (e.g., methyl esters) thereof, polymethacrylate and/or polyacrylate esters (e.g., poly(2-hydroxyethyl methacrylate).

As used herein, the phrase “ionic polymer” refers to a polymer is characterized by a charge density of at least 1 charged group (ionic group) per 1 kDa molecular weight of the polymer in an aqueous (e.g., water) environment at pH 7. The phrase “ionic polymer” encompasses polymers having a net negative charge (also referred to herein as “anionic polymers”), polymers having a net positive charge (also referred to herein as “cationic polymers”), and polymers having no net charge (also referred to herein as “zwitterionic polymers”), in an aqueous (e.g., water) environment at pH 7.

As used herein, the phrase “non-ionic hydrophilic polymer” encompasses any hydrophilic polymer (as defined herein) which is not an ionic polymer (as defined herein).

Herein throughout, the phrase “charged group” refers to any functional group (e.g., a functional group described herein) which is ionic (as defined herein), including, for example, amine, carboxylic acid, sulfate, sulfonate, phosphate and phosphonate. Thus, each electric charge in a moiety or molecule is associated with one charged group, although a single charged group (e.g., non-substituted phosphate) may be associated with more than one electric charge of the same sign (e.g., a dianion, a dication).

In some embodiments of any one of the embodiments described herein, at least 75% of the ionic groups in the ionic polymer have the same charge, that is, at least 75% of the ionic groups are cationic groups or are anionic groups, such that the ionic polymer is substantially cationic or anionic, respectively. In some embodiments, at least 90% of the ionic groups in the ionic polymer have the same charge. In some embodiments, at least 95% of the ionic groups in the ionic polymer have the same charge. In some embodiments, at least 98% of the ionic groups in the ionic polymer have the same charge. In some embodiments, at least 99% of the ionic groups in the ionic polymer have the same charge.

In some embodiments of any one of the embodiments described herein, about 50% of the ionic groups in the ionic polymer have a positive charge and about 50% of the ionic groups in the ionic polymer have a negative charge, such that the ionic polymer is substantially zwitterionic.

In some embodiments of any one of the embodiments described herein, the ionic polymer has from 1 to 6 charged groups per 1 kDa. In some embodiments, the ionic polymer has from 1.5 to 4 charged groups per 1 kDa. In some embodiments, the ionic polymer has from 2 to 3 charged groups per 1 kDa.

In some embodiments of any one of the embodiments described herein, the ionic polymer is characterized by a net charge (i.e., the difference between the number of anionic groups and the number of cationic groups) of from 1 to 6 electric charges per 1 kDa molecular weight of the polymer. In some embodiments, the ionic polymer has a net charge of from 1.5 to 4 charges per 1 kDa. In some embodiments, the ionic polymer has a net charge of from 2 to 3 charges per 1 kDa.

In some embodiments of any one of the embodiments described herein, the ionic polymer is an anionic polymer, for example, a polymer characterized by a net negative charge of from 1 to 6 electric charges per 1 kDa molecular weight of the polymer.

In some embodiments of any one of the embodiments described herein, the ionic polymer is a cationic polymer, for example, a polymer characterized by a net positive charge of from 1 to 6 electric charges per 1 kDa molecular weight of the polymer.

In some embodiments of any one of the embodiments described herein, the polymer is a polysaccharide. In some such embodiments, the polysaccharide is an ionic polymer. In some such embodiments, the polysaccharide is a non-ionic polymer.

As used herein, the term “polysaccharide” refers to a polymer composed primarily (at least 50 weight percents) of monosaccharide units linked by glycosidic linkages.

As used herein, the term “monosaccharide” encompasses carbohydrates per se (having the formula Cn(H₂O)n, wherein n is at least 3, typically from 3 to 10), as well as derivatives thereof such as amino sugars, in which at least one hydroxyl group is replaced by an amine or amide group; sugar acids, in which one or two carbon atoms are oxidized to form a carboxylate group; acylated monosaccharides, in which at least one hydroxyl group and/or amine group is substituted by an acyl group (e.g., acetyl); monosaccharide ethers (e.g., cellulose ethers), in which at least one hydroxyl group is substituted by an alkyl group (e.g., methyl, carboxymethyl, hydroxypropyl); and sulfated monosaccharides, in which at least one hydroxyl group is replaced by a sulfate group.

Examples of monosaccharides include, without limitation, hexoses (e.g., D-hexoses and/or L-hexoses) such as allose, altrose, glucose, mannose, gulose, idose, galactose, talose, psicose, fructose, sorbose and tagatose; pentoses (e.g., D-pentoses and/or L-pentoses) such as arabinose, lyxose, xylose, ribose, ribulose and xylulose; and hexose derivatives such as glucuronic acid, iduronic acid, mannuronic acid, guluronic acid, glucosamine and N-alkyl derivatives thereof, galactosamine and N-alkyl derivatives thereof, N-acetylglucosamine, N-acetylgalactosamine, and monosulfated and disulfated N-acetylgalactosamine, glucuronic acid and iduronic acid.

As used herein, the phrase “glycosidic linkage” refers to a bond between a hemiacetal group of one compound (e.g., a monosaccharide monomer) and a hydroxyl group of another compound (e.g., another monosaccharide monomer).

Examples of polysaccharides which are non-ionic polymers include, without limitation, dextran and cellulose derivatives such as hydroxypropyl methyl cellulose, hydroxypropyl cellulose, and methyl cellulose.

Examples of polysaccharides which are ionic polymers include, without limitation, carboxymethyl cellulose, hyaluronic acid, chondroitin sulfate, alginic acid, xanthan gum, chitosan and N-alkyl chitosan derivatives.

Hyaluronic acid is an anionic polysaccharide comprising anionic glucuronic acid monomer units along with non-ionic N-acetylglucosamine monomer units. Hyaluronic acid is an exemplary anionic polymer.

Chondroitin sulfate is an anionic polysaccharide comprising anionic sulfated (e.g., monosulfated and/or disulfated) N-acetylgalactosamine, glucuronic acid and/or iduronic acid monomer units, and anionic glucuronic acid and/or iduronic acid monomer units, along with non-ionic N-acetylgalactosamine monomer units.

Alginic acid is an anionic polysaccharide comprising anionic mannuronic acid and guluronic acid monomer units.

Xanthan gum is an anionic polysaccharide comprising anionic glucuronic acid monomer units, along with non-ionic glucose and mannose monomer units (including acetyl and/or pyruvyl derivatives thereof).

Chitosan is a cationic polysaccharide comprising cationic glucosamine monomer units, optionally along with non-ionic N-acetylglucosamine monomer units. In N-alkyl chitosan derivatives, at least a portion of the glucosamine units comprise 1, 2 or 3 alkyl groups, preferably C₁₋₄ alkyl, attached to the nitrogen atom. In some embodiments of any one of the embodiments described herein, the alkyl groups attached to the nitrogen atoms are each independently methyl or ethyl. In some embodiments, the alkyls are methyl. In some embodiments, the N-alkylated monomer unit is N-trimethylglucosamine.

Herein, the terms “hyaluronic acid”, “chondroitin sulfate”, “alginic acid”, “xanthan gum”, “chitosan”, “N-alkyl chitosan derivatives” and any other ionic compounds named herein, encompass all salts of the named compounds along with the non-ionic forms (e.g., acid forms of the anionic polysaccharides, and the free base forms of the cationic polysaccharides).

In some embodiments of any one of the embodiments described herein, the ionic polymer (e.g., ionic polysaccharide) is in a form of a salt. In some embodiments, the salt is a pharmaceutically acceptable salt (e.g., an ophthalmically acceptable salt for an ophthalmic application described herein).

In some embodiments of any one of the embodiments described herein relating to an ionic polymer which is a polysaccharide, the polysaccharide has from 0.2 to 1 charged groups per monosaccharide residue. In some embodiments, the polysaccharide has from 0.2 to 0.9 charged groups per monosaccharide residue. In some embodiments, the polysaccharide has from 0.3 to 0.7 charged groups per monosaccharide residue. In some embodiments, the polysaccharide has from 0.4 to 0.6 charged groups per monosaccharide residue. In some embodiments, the polysaccharide has about 0.5 charged groups per monosaccharide residue.

It is to be appreciated that a monosaccharide residue may comprise more than one charged group (e.g., a sulfate group and a carboxylate group).

In some embodiments of any one of the embodiments described herein relating to an ionic polymer which is a polysaccharide, the monosaccharide residues comprise no more than one charged group, that is, 0 or 1 charged group.

In some embodiments of any one of the embodiments described herein relating to an ionic polymer which is a polysaccharide, the polysaccharide is characterized by a net charge (i.e., the difference between the number of anionic groups and the number of cationic groups) of from 0.2 to 1 electric charges per monosaccharide residue. In some embodiments, the net charge is from 0.2 to 0.9 electric charges per monosaccharide residue. In some embodiments, the net charge is from 0.3 to 0.7 electric charges per monosaccharide residue. In some embodiments, the net charge is from 0.4 to 0.6 electric charges per monosaccharide residue. In some embodiments, the net charge is about 0.5 electric charges per monosaccharide residue.

In some embodiments of any one of the embodiments described herein, a molecular weight (e.g., weight averaged molecular weight) of the polymer is in a range of from 5 kDa to 10 MDa. In some embodiments, a molecular weight is from 10 kDa to 5 MDa. In some embodiments, a molecular weight is from 20 kDa to 2 MDa. In some embodiments, a molecular weight is from 50 kDa to 1 MDa.

In some embodiments of any one of the embodiments described herein, the molecular weight (weight averaged molecular weight) is from 5 kDa to 5 MDa. In some embodiments, a molecular weight is from 5 kDa to 2 MDa. In some embodiments, a molecular weight is from 5 kDa to 1 MDa. In some embodiments, a molecular weight is from 5 kDa to 500 kDa. In some embodiments, a molecular weight is from 5 kDa to 200 kDa. In some embodiments, a molecular weight is from 5 kDa to 100 kDa.

In some embodiments of any one of the embodiments described herein, the molecular weight (weight averaged molecular weight) is from 10 kDa to 10 MDa. In some embodiments, a molecular weight is from 20 kDa to 10 MDa. In some embodiments, a molecular weight is from 50 kDa to 10 MDa. In some embodiments, a molecular weight is from 100 kDa to 10 MDa. In some embodiments, a molecular weight is from 200 kDa to 10 MDa. In some embodiments, a molecular weight is from 500 kDa to 10 MDa.

In some embodiments of any one of the embodiments described herein, the polymer is selected to enhance an affinity of the liposomes to a surface of the substrate, that is, the liposome lipids have a greater affinity to the surface contacted with (e.g., coated by) the polymer than to the surface in the absence of the polymer.

In some embodiments of any one of the embodiments described herein relating to an ionic polymer, the ionic polymer is selected such that the liposomes are characterized by a surface charge having a sign opposite a sign of a net charge of the ionic polymer.

In some embodiments of any one of the embodiments described herein, the liposomes are characterized by a negative surface charge (e.g., as described herein in any one of the respective embodiments) and the ionic polymer has a net positive charge (e.g., as described herein in any one of the respective embodiments). In some embodiments, the ionic polymer is a polysaccharide having a net positive charge (e.g., a cationic polysaccharide described herein in any one of the respective embodiments).

In some embodiments of any one of the embodiments described herein, the liposomes are characterized by a positive surface charge (e.g., as described herein in any one of the respective embodiments) and the ionic polymer has a net negative charge (e.g., as described herein in any one of the respective embodiments). In some embodiments, the ionic polymer is a polysaccharide having a net negative charge (e.g., an anionic polysaccharide described herein in any one of the respective embodiments). In some embodiments, the ionic polymer is hyaluronic acid or alginic acid (optionally hyaluronate or alginate salts, in accordance with the definitions of “hyaluronic acid” and “alginic acid” used herein).

In some embodiments of any one of the embodiments described herein, the amphiphilic lipid of the liposomes comprises at least one phospholipid which comprises a phosphoethanolamine group or N-alkyl derivative thereof (e.g., in any one of the respective embodiments) and the ionic polymer has a net negative charge (e.g., as described herein in any one of the respective embodiments). In some embodiments, the lipid is a phosphatidylcholine (e.g., as described herein in any one of the respective embodiments). In some embodiments, the ionic polymer is a polysaccharide having a net negative charge (e.g., an anionic polysaccharide described herein). In some embodiments, the ionic polymer is hyaluronic acid or alginic acid.

In some embodiments of any one of the embodiments described herein, a concentration of polymer in a composition described herein is in a range of from 0.01 to 100 mg/ml. In some embodiments, the concentration is in a range of from 0.03 to 100 mg/ml. In some embodiments, the concentration is in a range of from 0.1 to 100 mg/ml. In some embodiments, the concentration is in a range of from 0.3 to 100 mg/ml. In some embodiments, the concentration is in a range of from 1 to 100 mg/ml. In some embodiments, the concentration is in a range of from 3 to 100 mg/ml. In some embodiments, the concentration is in a range of from 10 to 100 mg/ml.

In some embodiments of any one of the embodiments described herein, a concentration of polymer in a composition described herein is in a range of from 0.01 to 30 mg/ml. In some embodiments, the concentration is in a range of from 0.03 to 30 mg/ml. In some embodiments, the concentration is in a range of from 0.1 to 30 mg/ml. In some embodiments, the concentration is in a range of from 0.3 to 30 mg/ml. In some embodiments, the concentration is in a range of from 1 to 30 mg/ml. In some embodiments, the concentration is in a range of from 3 to 30 mg/ml.

In some embodiments of any one of the embodiments described herein, a concentration of polymer in a composition described herein is in a range of from 0.01 to 10 mg/ml. In some embodiments, the concentration is in a range of from 0.03 to 10 mg/ml. In some embodiments, the concentration is in a range of from 0.1 to 10 mg/ml. In some embodiments, the concentration is in a range of from 0.3 to 10 mg/ml. In some embodiments, the concentration is in a range of from 1 to 10 mg/ml.

In some embodiments of any one of the embodiments described herein, a concentration of polymer in a composition described herein is less than 3 mg/ml. In some embodiments, the concentration is at least 0.01 mg/ml. In some embodiments, the concentration is at least 0.03 mg/ml. In some embodiments, the concentration is at least 0.1 mg/ml. In some embodiments, the concentration is at least 0.3 mg/ml.

In some embodiments of any one of the embodiments described herein, a concentration of polymer in a composition described herein is less than 1 mg/ml. In some embodiments, the concentration is at least 0.01 mg/ml. In some embodiments, the concentration is at least 0.03 mg/ml. In some embodiments, the concentration is at least 0.1 mg/ml. In some embodiments, the concentration is at least 0.3 mg/ml.

In some embodiments of any one of the embodiments described herein, a concentration of polymer in a composition described herein is less than 0.5 mg/ml. In some embodiments, the concentration is at least 0.01 mg/ml. In some embodiments, the concentration is at least 0.03 mg/ml. In some embodiments, the concentration is at least 0.1 mg/ml.

Herein, for any one of the embodiments and for any combination thereof, a composition which comprises liposomes (optionally combined with a hydrogel and/or a polymer) is to be regarded as “anti-biofouling (ABF) composition”.

Contact Lenses and Keratitis:

According to another aspect of some embodiments of the invention, there is provided a method of inhibiting biofilm formation on a contact lens, the method comprising rinsing and/or immersing the contact lens in a solution comprising liposomes and an aqueous carrier. The method is optionally in accordance with a method of inhibiting biofilm formation on a surface of a substrate (according to any of the respective embodiments described herein), wherein the substrate is a contact lens.

In some embodiments of any one of the embodiments described herein, the method is for inhibiting formation of a biofilm of a pathogenic microorganism associated with keratitis.

According to another aspect of some embodiments of the invention, there is provided a method of treating keratitis in a contact-lens-wearing subject in need thereof, the method comprising rinsing and/or immersing a contact lens in a solution comprising liposomes and an aqueous carrier (according to any of the respective embodiments described herein), prior to insertion of the contact lens in an eye of the subject.

According to another aspect of some embodiments of the invention, there is provided a use of a solution comprising liposomes and an aqueous carrier in the manufacture of a medicament for the treatment of keratitis in a contact-lens-wearing subject in need thereof, the treatment comprising rinsing and/or immersing a contact lens in a solution comprising liposomes and an aqueous carrier (according to any of the respective embodiments described herein), prior to insertion of the contact lens in an eye of the subject.

According to another aspect of some embodiments of the invention, there is provided a solution comprising liposomes and an aqueous carrier (according to any of the respective embodiments described herein), for use in the treatment of keratitis in a contact-lens-wearing subject in need thereof, the treatment comprising rinsing and/or immersing a contact lens in a solution comprising liposomes and an aqueous carrier, prior to insertion of the contact lens in an eye of the subject.

Without being bound by any particular theory, it is believed that a solution which inhibits biofilm formation (e.g., according to any of the respective embodiments described herein) is useful in treating keratitis even if the solution has no direct effect on the responsible pathogen at the site of infection in the eye, as the inhibition of biofilm formation on the contact lens reduces the likelihood and/or degree of re-infection, thereby facilitating recovery, and/or reduces the risk of initiation of keratitis (e.g., as a prophylactic treatment). It is further believed that the pathogen at the site of infection may be removed by flow of tear fluid, an immune response, and or by therapy, for example, by action of a drug (e.g., administration of a suitable antimicrobial agent), and that the therapeutic efficacy of such removal of the pathogen is enhanced by inhibition of biofilm formation on the contact lens.

In some embodiments of any one of the embodiments described herein relating to keratitis, according to any of the aspects described herein, the keratitis is associated with contact lens use (e.g., diagnosed by a physician as being associated with contact lens use, for example, based on the symptoms of a contact lens user and/or on the infecting pathogen).

Examples of pathogenic microorganisms associated with keratitis (as well as keratitis which is associated with contact lenses) include, without limitation, amoebas (e.g., Acanthamoeba), bacteria (e.g., Staphylococcus aureus, Pseudomonas aeruginosa) and fungi (e.g., Fusarium).

In some embodiments of any one of the embodiments described herein, according to any of the aspects described herein, the solution further comprises at least one polymer. In some embodiments, the polymer is a hydrophilic polymer. In some embodiments, the hydrophilic polymer is a water-soluble polymer. In some embodiments, the water-soluble polymer is entirely dissolved, that is, the concentration of polymer is at or below the saturation point.

Any one of the embodiments described herein relating to a contact lens and/or keratitis may utilize liposomes in accordance with any one of the embodiments described herein with respect to liposomes and/or lipids (e.g., in the section herein relating to liposomes and lipids), as well as a polymer in accordance with any one of the embodiments described herein with respect to a polymer (e.g., in the section herein relating to polymers).

In some embodiments of any one of the embodiments described herein relating to a contact lens and/or keratitis, the liposomes are primarily (more than 50 weight percents) small unilamellar vesicles.

Without being bound by any particular theory, it is believed that inclusion of small unilamellar vesicles and/or dissolved polymer (as opposed to another form of liposome and/or polymer) allows for minimal effects on the transparency of the contact lens, due to the relatively low light scattering by small unilamellar vesicles and/or dissolved polymer molecules in comparison with larger particles.

In some embodiments of any one of the embodiments described herein relating to a contact lens and/or keratitis, the liposomes are characterized by a phase transition melting point (Tm) below 37° C. In some embodiments, the Tm is below 36° C. In some embodiments, the Tm is below 35° C. In some embodiments, the Tm is below 34° C. In some embodiments, the Tm is below 32° C. In some embodiments, the Tm is below 30° C. In some embodiments, the Tm is below 25° C. In some embodiments, the Tm is below 20° C.

In some embodiments of any one of the embodiments described herein, a concentration of polymer in the solution is in a range of from 0.01 to 10 mg/ml. In some embodiments, the concentration is in a range of from 0.03 to 10 mg/ml. In some embodiments, the concentration is in a range of from 0.1 to 10 mg/ml. In some embodiments, the concentration is in a range of from 0.3 to 10 mg/ml.

In some embodiments of any one of the embodiments described herein, a concentration of polymer in the solution is less than 3 mg/ml. In some embodiments, the concentration is at least 0.01 mg/ml. In some embodiments, the concentration is at least 0.03 mg/ml. In some embodiments, the concentration is at least 0.1 mg/ml. In some embodiments, the concentration is at least 0.3 mg/ml.

In some embodiments of any one of the embodiments described herein, a concentration of polymer in the solution is less than 1 mg/ml. In some embodiments, the concentration is at least 0.01 mg/ml. In some embodiments, the concentration is at least 0.03 mg/ml. In some embodiments, the concentration is at least 0.1 mg/ml. In some embodiments, the concentration is at least 0.3 mg/ml.

In some embodiments of any one of the embodiments described herein, a concentration of polymer in the solution is less than 0.5 mg/ml. In some embodiments, the concentration is at least 0.01 mg/ml. In some embodiments, the concentration is at least 0.03 mg/ml. In some embodiments, the concentration is at least 0.1 mg/ml. In some embodiments, the concentration is at least 0.3 mg/ml.

In some embodiments of any one of the embodiments described herein, a viscosity of the solution is no more than 1000 cP (centipoise). In some embodiments, the viscosity is no more than 500 cP. In some embodiments, the viscosity is no more than 200 cP. In some embodiments, the viscosity is no more than 100 cP. In some embodiments, the viscosity is no more than 50 cP. In some embodiments, the viscosity is no more than 20 cP. In some embodiments, the viscosity is no more than 10 cP. In some embodiments, the viscosity is no more than 5 cP. In some embodiments, the viscosity is no more than 3 cP. In some embodiments, the viscosity is no more than 2 cP.

Herein, viscosities of a solution are determined at a temperature of 20° C. and at a shear rate of 1 second⁻¹ (unless indicated otherwise).

In some embodiments of any one of the embodiments described herein, the carrier is an ophthalmically acceptable carrier. In some such embodiments, the solution can be allowed to remain on the contact lens following rinsing and/or immersing in the solution, as the residual solution will not harm the eye when the contact lens is placed on the eye.

Herein, the phrase “ophthalmically acceptable carrier” refers to a carrier or a diluent that does not cause significant irritation to a subject when contacted with an eye (e.g., cornea and/or sclera) of the subject, and does not abrogate the activity and properties of the liposomes (and polymer, if present) in the solution (e.g., their ability to inhibit biofilm formation on a contact lens surface).

In some embodiments of any one of the embodiments described herein, the carrier is not an ophthalmically acceptable carrier. Examples of such carriers include, without limitation, carriers comprising a preservative and/or a concentration of preservative which is not ophthalmically acceptable. Such carriers may be suitable, for example, for immersing a contact lens for an extended period of time, and/or for storage for an extended period of time, while limiting the risk of bacterial growth in the solution. Typically, a solution comprising such a carrier is rinsed with an ophthalmically acceptable liquid (e.g., water, saline) solution prior to placing the contact lens on the eye.

In some embodiments of any one of the embodiments described herein, the solution comprises a buffer, (e.g., borate and/or phosphate), for example, such that the solution has a pH of from about 6.5 to 7.6).

In some embodiments of any one of the embodiments described herein, the solution is formulated as a solution suitable for storage of contact lenses (e.g., hydrogel contact lenses), for example, as known in the art.

In some embodiments of any one of the embodiments described herein, a contact lens is packaged in a packaging material (e.g., a blister pack) and immersed in the solution contained within a packaging material. In some embodiments, the contact lens is a hydrogel contact lens, and the solution is a solution suitable for storage of hydrogel contact lenses, for example, as known in the art.

Without being bound by any particular theory, it is believed that packaging a contact lens immersed in the solution comprising liposomes (optionally with a polymer, as described herein) provides ample time for the liposome lipids (and optionally also the polymer) to coat the contact lens surface, as described herein in any one of the respective embodiments, and avoids the need for a user of the contact lens to contact the contact lens with the solution.

In some of any one of the embodiments described herein which relate to a contact lens, according to any one of the aspects described herein, the contact lens comprises a hydrogel surface. In some embodiments, the contact lens comprises a hydrogel surface and a rigid center. In some embodiments, the contact lens consists essentially of a hydrogel.

The hydrogel may comprise any material known in the art for use in contact lens hydrogels. Examples of such hydrogel materials include, without limitation, alphafilcon (e.g., alphafilcon A), asmofilcon (e.g., asmofilcon A), balafilcon (e.g., balafilcon A), bufilcon (e.g., bufilcon A), comfilcon (e.g., comfilcon A), crofilcon, deltafilcon (e.g., deltafilcon A), dimefilcon, droxifilcon (e.g., droxifilcon A), enfilcon (e.g., enfilcon A), etafilcon (e.g., etafilcon A), filcon II, galyfilcon (e.g., galyfilcon A), hefilcon (e.g., hefilcon A, hefilcon B), hilafilcon (e.g., hilafilcon A, hilafilcon B), hioxifilcon (e.g., hioxifilcon A, hioxifilcon D), isofilcon, lidofilcon (e.g., lidofilcon A, lidofilcon B), lotrafilcon (e.g., lotrafilcon B), mafilcon, methafilcon (e.g., methafilcon A, methafilcon B), narafilcon (e.g., narafilcon A, narafilcon B), nelfilcon (e.g., nelfilcon A), ocufilcon (e.g., ocufilcon A, ocufilcon B), ofilcon (e.g., ofilcon A), omafilcon (e.g., omafilcon A), perfilcon, phemfilcon (e.g., phemfilcon A), polymacon, scafilcon (e.g., scafilcon A), senofilcon (e.g., senofilcon A), surfilcon, tefilcon, tetrafilcon (e.g., tetrafilcon A, tetrafilcon B), vifilcon (e.g., vifilcon A), and xylofilcon (e.g., xylofilcon A).

In some embodiments of any one of the embodiments described herein, the hydrogel comprises a polymer selected from the group consisting of poly(2-hydroxyethyl methacrylate) (e.g., cross-linked poly(2-hydroxyethyl methacrylate), polyvinyl alcohol (e.g., cross-linked polyvinyl alcohol) and a silicone. In some embodiments, the polymer comprises a silicone. Such polymers may optionally comprise small amounts of additional monomers (e.g., cross-linking monomers) copolymerized with the 2-hydroxyethyl methacrylate, polyvinyl alcohol or silicone monomer. For example, 2-hydroxyethyl methacrylate may optionally be copolymerized with vinylpyrrolidone, methyl methacrylate, methacrylic acid (an anionic monomer), ethylene glycol dimethacrylate (a cross-linking monomer) and/or 3-(ethyldimethyl-ammonium)propyl methacrylamide (a cationic monomer) in a contact lens hydrogel.

In some embodiments of any one of the embodiments described herein, the hydrogel of the contact lens consists essentially of a polymer which forms a hydrogel (e.g., a cross-linked polymer) and an aqueous liquid (optionally water).

It is expected that during the life of a patent maturing from this application many relevant contact lens and hydrogels for forming contact lens will be developed and the scope of the terms “contact lens” is intended to include all such new technologies a priori.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a polymer” or “at least one polymer” may include a plurality of polymers, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non-limiting fashion.

### Materials and Methods

Materials:

Alginic acid (from brown algae; sodium salt; medium viscosity) was obtained from Sigma-Aldrich.

Dimyristoyl phosphatidylcholine (DMPC) was obtained from Lipoid GmbH.

Gelatin (type A from porcine skin) was obtained from Sigma-Aldrich.

Hyaluronic acid (from rooster comb; sodium salt) was obtained from Sigma-Aldrich.

Hydroxypropyl methyl cellulose (HPMC; 2,600-5,600 cP at a concentration of 2% in water at 20° C.) was obtained from Sigma.

Polyethylene oxide (PEO; average molecular weight of 200 kDa) was obtained from Sigma-Aldrich.

Polyethylene oxide dimethacrylate (PEOdMA; molecular weight 550 Da) was obtained from Sigma-Aldrich.

Poly(2-hydroxyethyl methacrylate) (polyHEMA; average molecular weight of 20 kDa) was obtained from Sigma-Aldrich.

Polyvinylpyrrolidone (PVP; average molecular weight of 40 kDa) was obtained from Sigma-Aldrich. Distilled water was purified in a Barnsted NanoPure system to 18.2 MS) cm resistance with total organic content levels of less than approximately 1 part per billion.

All other compounds were obtained from Sigma-Aldrich.

Hydrogel Preparation:

Neat HEMA (2-hydroxyethyl methacrylate) hydrogels, containing 2% (by molar ratio of monomers) EGDMA (ethylene glycol dimethacrylate) cross-linker (unless a different cross-linker concentration is indicated), were prepared as follows: HEMA (3.2 grams), EGDMA (100 mg for preparing a concentration of 2%) and APS (ammonium persulfate) aqueous solution (2 ml, 53 mM) were vigorously stirred for 30 minutes until fully mixed. 50 μl TMEDA (N,N,N′,N′-tetramethylethylenediamine) was added to the mixture, stirred for 20 seconds and poured into a 6 cm diameter petri dish. The gels were allowed to cross-link over 4-5 hours, followed by rinsing in distilled water for 3 days to remove unreacted materials. The obtained gels were cut into pieces for protein adsorption tests.

Liposome-loaded HEMA hydrogels, polymer-loaded hydrogels and liposome/polymer-loaded hydrogels were prepared by replacing the APS aqueous solution with a liposome or a liposome/polymer suspension with the same APS content.

MAA-PEO (methacrylamide-polyethylene oxide) hydrogels with an initial concentration of 31 weight percents methacrylamide and 15.7 weight percents PEOdMA (polyethylene oxide dimethacrylate) were prepared by photo-initiated free radical cross-linking in the presence of the water-soluble photoinitiator Irga 2959 (0.3 weight percent). The hydrogel solutions were stirred until the solution turned clear, and then they were poured into a 6 cm-diameter glass petri dish. The samples were cured by exposure to UV light for 15 minutes. For preparation of hydrogels containing liposomes, pure water was replaced by a DMPC (dimyristoyl phosphatidylcholine) multilamellar vesicle (MLV) solution with a concentration of 39 mM.

Gelatin methacrylate (GM) was synthesized according to procedures as previously described [van den Bulcke et al., Biomacromolecules 2000, 1:31-38; Hoch et al., J Mat Chem B 2013, 1:5675-5685]. Gelatin (4 grams) was dissolved in phosphate buffer (40 ml, pH 7.4) at 40° C. The pH of the solution was then adjusted to 7.5 using an NaOH solution. 2 ml of methacrylic anhydride was added drop-wise after the gelatin was completely dissolved. During the methacrylation reaction the mixture was stirred and the pH of the solution was kept within the range of 7.0-7.5 for two hours of reaction time at 50° C. Then, the mixture was diluted with PBS and dialyzed for 2 days against distilled water at 40° C. The reaction product was freeze-dried and stored at −20° C. until use.

Contact Lenses:

The following commercial hydrogel soft contact lenses were used, and their composition, water content and modulus are summarized below. Narafilcon A (Johnson & Johnson, 1 Day TruEye®), nelfilcon A (Ciba Vision, Focus® Dailies®) and filcon II (Sauflon UK) contact lenses were originally immersed in saline solution in their as-purchased blister pack.

| | Composition | Water | Lens |
|---|---|---|---|
| Component | (monomers) | content | modulus |
| Narafilcon A | Silicone | 46% | 0.66 MPa |
| Methafilcon | HEMA, MA | 55% | |
| Nelfilcon A | Vinyl alcohol | 69% | |
| Filcon II | Silicone | 56% | 0.5 MPa |

HEMA= 2-hydroxylethyl methacrylate;
MA = methacrylic acid

Liposome and Liposome/Polymer Mixture Preparation:

Multilamellar vesicles (MLV) composed of dimyristoyl phosphatidylcholine (DMPC; 1,2-dimyristoyl-sn-glycero-3-phosphocholine) were prepared by hydrating the lipids at least 5° C. above the lipid melting point (T_{M}), followed by sonication in PBS (phosphate buffer saline).

Small unilamellar vesicles (SUV) of approximately 70 nm in diameter were formed by downsizing MLVs by stepwise extrusion through polycarbonate membranes starting with a 400-nm and ending with 50-nm-pore-size membrane, using a Lipex 10 ml extruder system (Northern Lipids, Vancouver, Canada).

When liposomes were mixed with polymer, the polymer solution was prepared in advance in PBS, and after full dissolution of the polymer, the solution was warmed to at least 5° C. above the lipid T_{M}, and added to the lipids, followed by mixing by stirring.

Protein Adsorption Measurements:

Nonspecific protein adsorption to the hydrogels was evaluated using horseradish peroxidase (HRP)-conjugated anti-IgG adsorption, using tissue culture polystyrene (TCPS) and contact lenses soaked in PBS as controls. The samples were incubated with 1 μg/ml HRP-conjugated anti-IgG for 2 hours in a 24-well plate at 37° C. and a shaking velocity of 200 rotations per minute, followed by five rinses with phosphate-buffered saline (PBS) buffer. The samples were then transferred to new wells. 1 ml of 1 mg/ml o-phenylenediamine (OPD) in 0.1 M citrate-phosphate buffer (pH 5.0) containing 0.03% hydrogen peroxide was added. Enzyme activity was stopped by adding an equal volume of 4 M H₂SO₄ after 15 minutes. The resulting tangerine color was measured at a wavelength of 492 nm in a 96-well plate.

### Example 1

### Protein Adsorption by Hydrogels Loaded with Liposomes and/or Polymers

Hydrogels were prepared from HEMA (2-hydroxyethyl methacrylate), MAA-PEO (methacrylamide-polyethylene oxide) or gelatin methacrylate, with or without DMPC or HSPC liposomes, as described in the Materials and Methods section hereinabove. Protein adsorption on the hydrogels was measured using anti-IgG antibodies as the protein, as described hereinabove, with results being normalized relative to adsorption on tissue culture polystyrene (TCPS) surfaces.

In order to evaluate the effect of various polymers on protein adsorption, HEMA hydrogels were prepared loaded with hyaluronic acid (HA), alginate, gelatin, hydroxypropyl methyl cellulose (HPMC), polyvinylpyrrolidone (PVP), polyethylene oxide (PEO) or non-cross-linked poly(2-hydroxyethyl methacrylate) (polyHEMA), with or without DMPC liposomes. It is to be appreciated that the loaded non-cross-linked polyHEMA differs from the polyHEMA cross-linked with EGDMA (ethylene glycol dimethacrylate) from which the HEMA hydrogel is formed.

As shown in FIG. 1, protein adsorption on a HEMA hydrogel with DMPC liposomes was about 4-fold lower than protein adsorption on a neat HEMA hydrogel (without liposomes), and more than 10-fold lower than protein adsorption on a tissue culture polystyrene surface.

As further shown therein, incorporation of the polymers alginate, HA, HPMC, PVP or PEO in a HEMA hydrogel with DMPC liposomes also resulted in reduced protein adsorption, with incorporation of HPMC, PVP or PEO further reducing protein adsorption by about 50% as compared to HEMA with DMPC liposomes alone.

As further shown in FIG. 1, HEMA hydrogels loaded with HSPC liposomes and HPMC reduced protein adsorption in comparison with both unloaded HEMA hydrogel and HEMA hydrogel loaded with HMPC without liposomes. This result indicates that HSPC liposomes, which were in a solid phase, can reduce protein adsorption, although the reduction in protein adsorption may be to a lesser degree than that obtained with DMPC liposomes, which were in a liquid phase.

In order to evaluate the effect of hydrogel cross-linking on protein adsorption, HEMA hydrogels were prepared with 1%, 2% or 4% EGDMA cross-linker and loaded with DMPC MLVs and/or non-cross-linked polyHEMA.

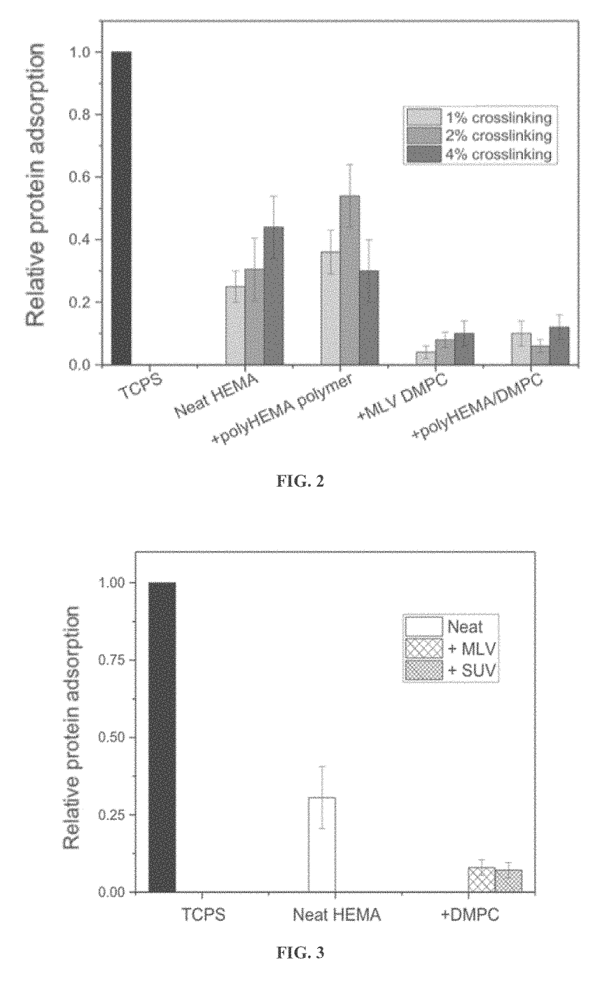

As shown in FIG. 2, DMPC liposomes loaded into HEMA hydrogels (alone or along with non-cross-linked polyHEMA) considerable reduced protein adhesion to hydrogels with 1%, 2% or 4% cross-linker. These results indicate that the effect of liposomes on adhesion to hydrogels occurs at a variety of degrees of hydrogel cross-linking.

In order to evaluate the effect of liposome type on protein adsorption, HEMA hydrogels loaded with DMPC multilamellar vesicles (MLVs) were compared with HEMA hydrogels loaded with DMPC small unilamellar vesicles (SUVs).

As shown in FIG. 3, DMPC MLVs and DMPC SUVs both considerably reduced protein adhesion to HEMA hydrogels. These results indicate that different types of liposomes are effective at reducing adhesion.

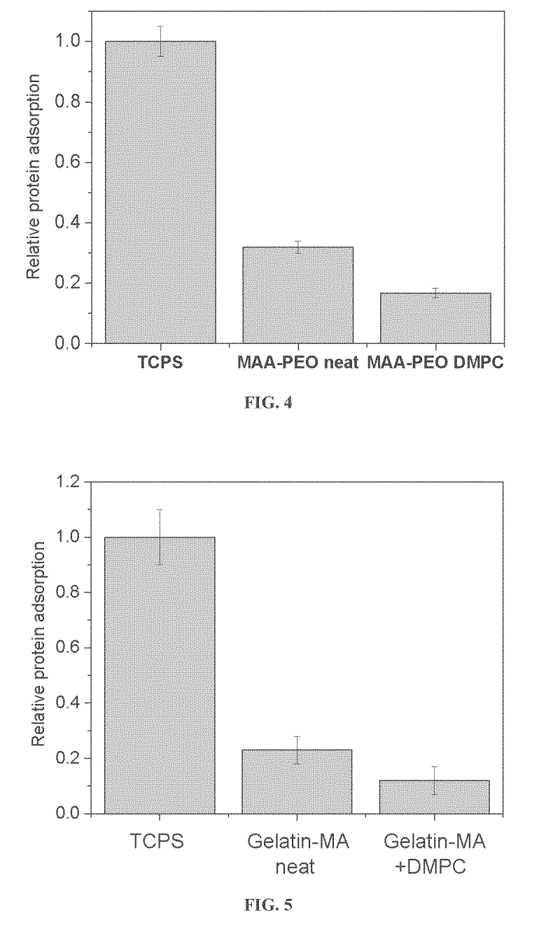

As shown in FIG. 4, protein adsorption on a MAA-PEO hydrogel with DMPC liposomes was lower by about 50% than protein adsorption on a neat MAA-PEO hydrogel (without liposomes), and about 6-fold lower than protein adsorption on a tissue culture polystyrene surface.

As shown in FIG. 5, protein adsorption on a gelatin methacrylate hydrogel with DMPC liposomes was lower by about 50% than protein adsorption on a neat gelatin methacrylate hydrogel (without liposomes), and about 8-fold lower than protein adsorption on a tissue culture polystyrene surface.

These results indicate that incorporation of liposomes in hydrogels reduces protein adsorption on the hydrogels, and that this effect of liposomes may be synergistically enhanced by incorporation of polymers such as HPMC, PVP and PEO.

The above results further indicate that the abovementioned effects occur on a variety of hydrogel types, including different species of polymer and different degrees of hydrogel cross-linking.

It is believed that the reduction in protein adsorption by liposomes is associated with a hydration layer formed on the gel surface by exposed phosphocholine groups of the liposomes.

In order to assess the resilience of the inhibitory effect on adsorption, HEMA hydrogels with various degrees of cross-linking, with and without loaded liposomes and/or polymers, were subjected to complete drying, followed by rehydration in PBS. Protein adsorption on the rehydrated hydrogels was then evaluated as described hereinabove.

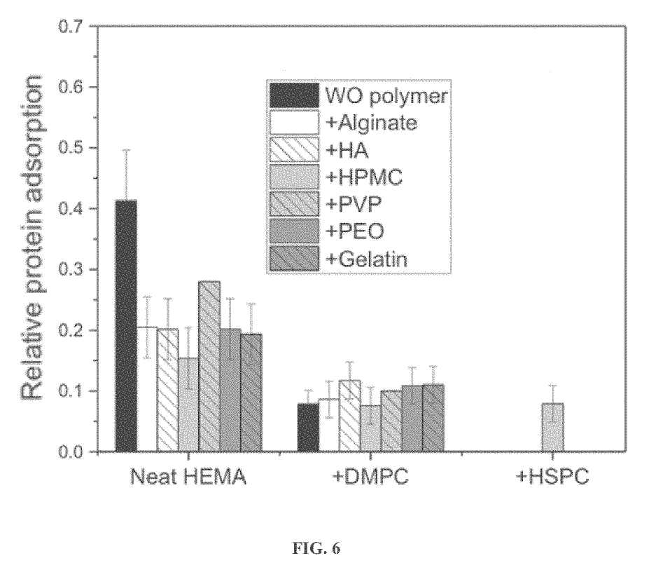

As shown in FIG. 6, protein adsorption on a dried and rehydrated HEMA hydrogel loaded with DMPC liposomes (with or without alginate, HA, HPMC, PVP, PEO or gelatin) or with HSPC liposomes and HPMC was about 4-fold lower than protein adsorption on a rehydrated neat HEMA hydrogel (without liposomes) and about 10-fold lower than protein adsorption on a tissue culture polystyrene surface. These results are similar to those obtained without drying and rehydration of the hydrogel (see FIG. 1).

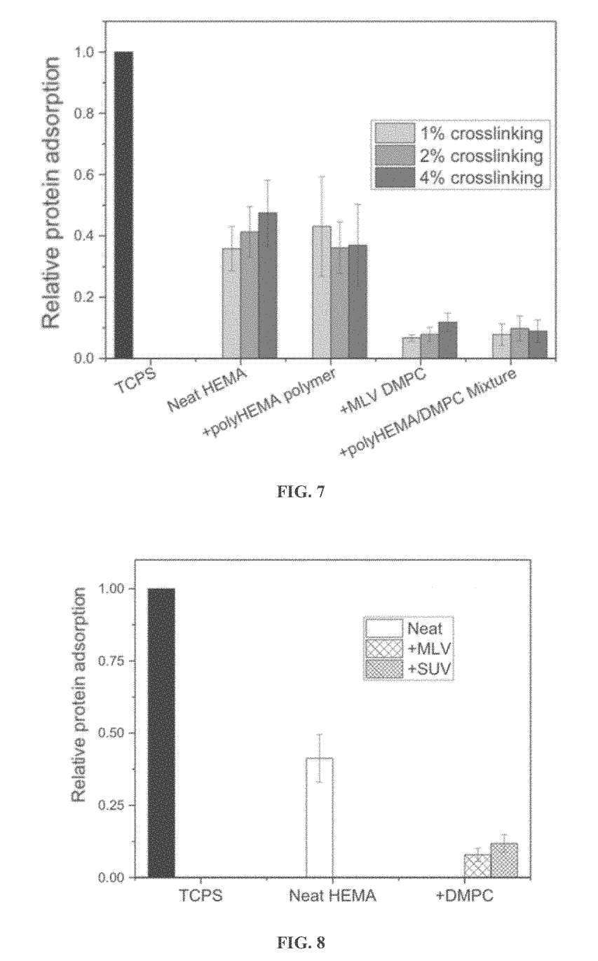

As shown in FIG. 7, protein adsorption on a dried and rehydrated HEMA hydrogel with 1%, 2% or 4% cross-linker, loaded with DMPC liposomes (with or without non-cross-linked polyHEMA), was considerably lower than protein adsorption on a rehydrated neat HEMA hydrogel (without liposomes).

As shown in FIG. 8, DMPC MLVs and DMPC SUVs both considerably reduced protein adhesion to dried and rehydrated HEMA hydrogels.

These results indicate that considerable inhibition of adsorption can be obtained even after dehydration and rehydration of liposome-loaded hydrogels.

### Example 2

### Protein Adsorption by Contact Lenses Treated with Solution of Liposomes and Polymer

Soft contact lenses made from various materials (narafilcon A, nelfilcon, filcon II and methafilcon A) were removed from saline solution (in their original containers) and placed for 72 hours in a solution of a polymer (hyaluronic acid or hydroxypropyl methyl cellulose) and/or dimyristoyl phosphatidylcholine (DMPC) liposomes, or in a PBS (phosphate buffer saline) control solution. Protein adsorption on the contact lenses was measured using anti-IgG antibodies as the protein, as described in the Materials and Methods section hereinabove.

In one experiment, contact lenses made from narafilcon A, a silicone hydrogel, were immersed in a solution of 0.1 mg/ml hyaluronic acid (HA) and DMPC liposomes in the form of multilamellar vesicles (MLVs), at a DMPC concentration of 2.5 mM.

As shown in FIG. 9, protein adsorption on narafilcon A contact lenses immersed in hyaluronic acid and DMPC liposomes was reduced by 40% relative to contact lenses immersed in PBS, whereas immersion in a solutions of DMPC liposomes alone reduced protein adsorption by 28%, and immersion in a solution of hyaluronic acid reduced protein adsorption by 13%.

In another experiment, nelfilcon, filcon II and methafilcon A contact lenses were immersed in a solution of 0.2 mg/ml hydroxypropyl methyl cellulose (HPMC) and DMPC liposomes in the form of small unilamellar vesicles (SUVs), at a DMPC concentration of 10 mM.

As shown in FIG. 10, protein adsorption on nelfilcon and filcon II contact lenses immersed in HPMC and DMPC liposomes was reduced by about 30% relative to contact lenses immersed in PBS, and protein adsorption on methafilcon A contact lenses immersed in HPMC and DMPC liposomes was reduced by about 60% relative to contact lenses immersed in PBS.

These results indicate that contact of a surface, such as a contact lens surface, with a polymer and liposomes can reduce protein adsorption on the surface.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1. A method of inhibiting adsorption of a biofouling-promoting agent on a surface of a substrate and/or inhibiting biofilm formation on a surface of a substrate, the method comprising contacting the substrate with a composition which comprises liposomes.

2. The method of claim 1, wherein said biofouling-promoting agent is selected from the group consisting of a biofouling-promoting protein and a biofouling-promoting polysaccharide.

3. The method of claim 1, wherein said liposomes are characterized by a melting point below a temperature of said surface during said contacting.

4. The method of claim 1, wherein said substrate is composed of a hydrogel.

5. The method of claim 4, wherein said contacting comprises incorporating said composition which comprises liposomes within said hydrogel.

6. The method of claim 4, wherein said substrate is prepared by forming said hydrogel in the presence of said liposomes.

7. (canceled)

8. The method of claim 1, wherein said composition further comprises a hydrogel.

9-10. (canceled)

11. The method of claim 1, wherein said adsorption is reduced by at least 30% relative to adsorption on said surface of said substrate in the absence of said composition which comprises liposomes.

12-27. (canceled)

28. The method of claim 1, wherein said biofilm formation is reduced by at least 10% relative to biofilm formation on said surface of said substrate in the absence of said composition which comprises liposomes.

29. The method of claim 1, wherein said composition which comprises liposomes further comprises a polymer.

30. A composition comprising liposomes, identified for use in inhibiting biofilm formation on a surface of a substrate and/or in inhibiting adsorption of a biofouling-promoting agent on a surface of a substrate.

31. (canceled)

32. The composition of claim 30, being in a form of a hydrogel comprising said liposomes.

33. (canceled)

34. The composition of claim 30, further comprising a polymer.

35-36. (canceled)

37. An article comprising a substrate and the composition of claim 30 being applied on a surface of the substrate.

38. The article of claim 37, being characterized in that under biofouling-promoting conditions, a biofilm load on the substrate is lowered by at least 10% compared to a biofilm load on said substrate when being devoid of said composition, under said conditions.

39. The method of claim 1, wherein said substrate is a contact lens, the method comprising rinsing and/or immersing the contact lens in a solution comprising liposomes and an aqueous carrier.

40. (canceled)

41. A method of treating keratitis in a contact lens-wearing subject in need thereof, the method comprising rinsing and/or immersing a contact lens in a solution comprising liposomes and an aqueous carrier, prior to insertion of said contact lens in an eye of the subject.

42. The method of claim 41, wherein said liposomes are characterized by a melting point below 36° C.

43. The method of claim 41, wherein said solution further comprises at least one polymer.

44-50. (canceled)

51. The composition of claim 30, being devoid of an antimicrobial agent.
